(19) United States
(12) Patent Application Publication
(10) Pub. No.: US 2012/0077722 A1
(43) Pub. Date: Mar. 29, 2012

(54) AMBERGRIS FRAGRANCE

(75) Inventors: Erich Dilk, Holzminden (DE); Marcus Eh, Holzminden (DE); Horst Surburg, Holzminden (DE)

(73) Assignee: SYMRISE AG, Holzminden (DE)

(21) Appl. No.: 13/247,479

(22) Filed: Sep. 28, 2011

Related U.S. Application Data
(60) Provisional application No. 61/387,560, filed on Sep. 29, 2010.

(51) Int. Cl. A61K 8/49; A61L 9/01; C11D 3/50; C07D 317/70; A61Q 13/00

(52) U.S. Cl. 510/103; 549/432; 512/12

(57) ABSTRACT

The present invention relates primarily to the novel compound of the formula (II) and certain mixtures comprising the compound of the formula (II) and use thereof as perfume. In addition the invention relates to perfume mixtures and perfumed products comprising a (preferably sensorially effective) amount of the compound of the formula (II) or a mixture according to the invention.

The present invention relates primarily to the novel compound of formula (II) and certain mixtures comprising the compound of formula (II) and use thereof as perfume. In addition the invention relates to perfume mixtures and perfumed products comprising a (preferably sensorially effective) amount of the compound of formula (II) or a mixture according to the invention. Further aspects of the invention can be seen from the following description and the appended patent claims.

Cedrane derivatives of formula (A) are described generally in EP 857 723 A1 and their odor properties are presented. According to this, compounds of this substance class possess an odor of the ambergris type and at the same time impart a glorious, strong effect, which intensifies quite varied perfume notes and prolongs their fragrant effect. However, no concrete descriptions of odor were undertaken for the compounds given in examples 5-17.

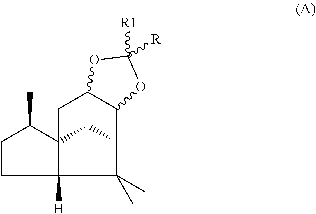

The wavy lines can signify alpha- or beta-configuration, in each case independently of one another.

For compounds in which R is different from R¹, no statements are made regarding the odor properties of the respective stereoisomers. In EP 857 723 A1 it is merely stated that the acetals of formula (A) in each case in pure form and as mixture of stereoisomers, possess original perfume properties.

The compound of formula (A1) [(4aR,5R,7aS,9R)-octahydro-2,2,5,8,8,9a-hexamethyl-4H-4-a,9-methanoazuleno(5,6-d)-1,3-dioxol, CAS No. 211299-54-6; hereinafter: Ambrocenide®] has the following structure, cf. EP 0 857 723 A1:

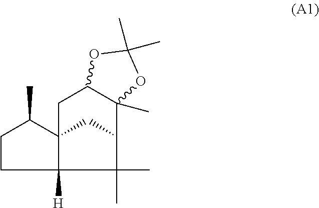

The wavy lines can signify, independently of one another, alpha- or beta-configuration. Ambrocenide® of formula (A1) can comprise one, two, three or all of the following diastereoisomers:

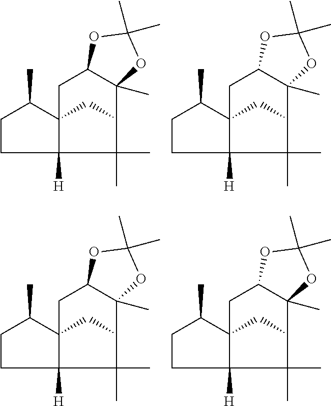

For a substance to be used as a perfume, in addition to an interesting odor profile, some other properties of the substance are also important, e.g. stability, compatibility with other perfumes, solubility, and toxicological harmlessness.

Further interesting aspects are furthermore the adherence or the substantivity of a perfume, in particular on fibers and/or the hair. This property is particularly important for products containing surfactants, such as shampoos, detergents and fabric softeners.

Ambrocenide® is an interesting perfume with pronounced ambergris odor and notably strong intrinsic adhesion.

Ambergris perfumes with strong intrinsic adhesion are required, in particular for the perfuming of surfactant-containing formulations. Preferably the Ambergris perfumes should have valuable odor notes. In this connection, the odor note of white amber is especially desirable. “White amber” means, in perfumery terms, the odor of aged natural ambergris, which represents a very valuable odor note.

Therefore the object to be solved by the present invention was to provide a substance (mixture or individual compound) with the ambergris odor and especially strong intrinsic adhesion, which preferably has an odor note of white amber.

This object is solved according to the invention by the compound of the following formula (II) and certain mixtures comprising compound (II) and compound (I).

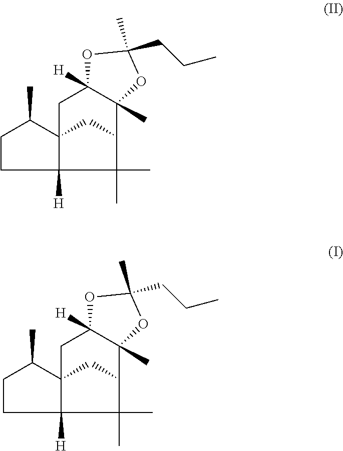

It was found, surprisingly, that the stereoisomer of formula (II) provides a solution to this object. The compound of formula (II) is characterized by a strong odor of ambergris and wood.

During olfactory evaluation of the stereoisomeric compounds of formulae (I) and (II), surprisingly only the compound according to the invention of formula (II) was found to be of olfactory interest, whereas the isomeric compound of formula (I) only had a very faint odor.

The compound of formula (II) can be used as a perfume both alone or mixed with the compound of formula (I).

During olfactory evaluation of the compound of formula (II) it was also found that this compound possesses exceptionally long adherence, even exceeding that of Ambrocenide®.

The acetals of formulae (Z-1) and (Z-2), which are related structurally to compounds (I) and (II), were obtained from cis-cedranediol and methyl ethyl ketone (2-butanone).

The compound of formula (Z-2) has a much stronger odor than the isomeric compound of formula (Z-1).

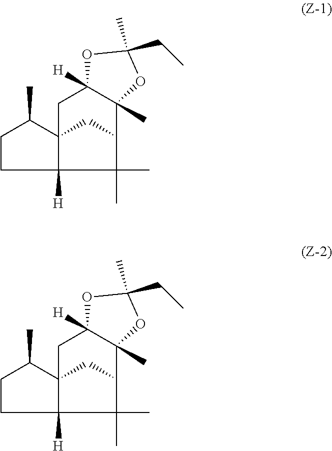

An olfactory comparison of the compounds of formula (II) and (Z-2) showed that the compound according to the invention of formula (II) has a much stronger odor.

The invention also relates to a mixture, comprising

(i) compound of formula (I)
and
(ii) compound of formula (II),
wherein
the weight ratio of the compound of formula (II) to the compound of formula (I) is in the range from 4:1 to 1:10, preferably in the range from 2:1 to 1:6, more preferably in the range from 1:1 to 1:3,
   and/or
the total proportion of the compounds of the formulae (I) and (II) together is greater than 40 wt. %, preferably greater than 55 wt. %, more preferably greater than 70 wt. %, even more preferably greater than 85 wt. %, and especially preferably greater than 95 wt. %, relative to the total amount of 3,6,8,8-tetramethyl-octahydro-3a,7-methano-azulene-5,6-diol-methyl-n-propyl ketals contained in the mixture.

The compound according to the invention of the formula (II) can be produced by reacting cis-cedranediol (compound of formula (B), which can be prepared from (−)-α-cedrene) with methyl-n-propyl-ketone (2-pentanone, compound of the formula (C)).

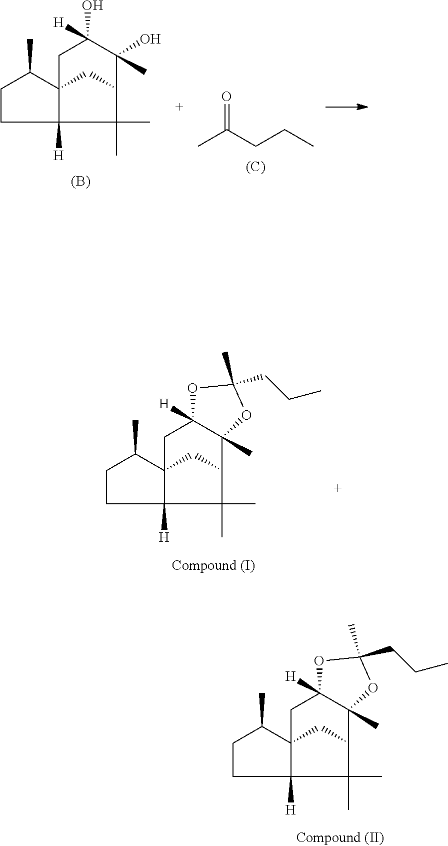

The compound according to the invention of the formula (II) or a mixture according to the invention (as defined above) can be produced by reacting cis-cedranediol (formula (B)) with methyl-n-propyl-ketone (2-pentanone) in the presence of one or a plurality of water-binding substances and one or a plurality of acids.

Preferred water-binding substances are selected from the group consisting of orthoformic acid esters (preferably orthoformic acid trimethyl ester or orthoformic acid triethylester) and 2,2-dialkoxypentanes (preferably 2,2-dimethoxypentane or 2,2-diethoxypentane).

The acids used are preferably selected from the group consisting of inorganic protic acids, organic protic acids and fixed-bed acid catalysts.

The reaction of cis-cedranediol (formula (B)) with methyl-n-propyl-ketone (2-pentanone) to the compound according to the invention of formula (II) or a mixture according to the invention (as defined above) preferably takes place at a reaction temperature in the range from 0 to 70° C.

The compound according to the invention of formula (II) or a mixture according to the invention (as defined above) is characterized by a high affinity (intrinsic adhesion on a substrate) and a high substantivity (ability to be absorbed from a, generally aqueous, phase onto a substrate or to remain on a substrate after a washing or rinsing operation). This effect is seen in particular on substrates such as skin, hair and textile fibers (e.g. wool, cotton, linen, synthetic fibers).

A further important application requirement for perfumes and perfume mixtures, in particular for products containing surfactants, is their substantivity with respect to or retention on the substrate, in particular hair or textile fibers. The terms “substantivity” and “retention” are explained in detail e.g. in EP 1 201 738 A1, cf. sections [0004]-[0005]. Perfumes with high substantivity and/or retention are generally required.

Based on this knowledge, a further aspect of the invention relates in particular to the use of the compound according to the invention of formula (II) or a mixture according to the invention (as defined above) as an agent for increasing the substantivity and/or retention of a perfume mixture (in particular with respect to or on hair or textile fibers), preferably a perfume mixture with a fruity, flowery, and/or spicy note.

As well as its high affinity, the compound according to the invention of formula (II) is characterized by its fixing properties, i.e. it is a fixative. As a fixative, the compound according to the invention of formula (II) increases the substantivity of other perfumes, either by lowering its vapor pressure or olfactory intensification (e.g. lowering the threshold value). The invention therefore—as already mentioned—also relates to the use of the compound according to the invention of the formula (II) or a mixture according to the invention (as characterized above) as fixative.

The invention therefore further relates to the use of the compound according to the invention of the formula (II) or of a mixture according to the invention as defined above
as perfume, preferably as perfume with the odor notes ambergris and/or wood,
   and/or
as an agent for increasing the substantivity and/or the retention of a perfume mixture,
   and/or
as fixative.

Furthermore, the invention relates to a perfume mixture, preferably a perfume oil, comprising
the compound according to the invention of the formula (II) or a mixture according to the invention (as defined above),
   and
one, 2, 3, 4, 5, 6, 7, 8, 9, 10 or a plurality of further perfumes, which are not 3,6,8,8-tetramethyl-octahydro-3a,7-methano-azulene-5,6-diol-methyl-n-propyl ketals,
   wherein
   (i) the amount of compound of the formula (II) is sufficient to impart an ambergris note and/or woody note, in particular a white amber note,
   and/or
   (ii) the further perfume(s) impart one, a plurality of or all of the notes fruity, flowery, spicy, woody, musk and/or ambergris-like and the amount of the compound of the formula (II) is sufficient to modify and/or intensify one or a plurality of these notes,
   and/or
   (iii) the amount of compound of the formula (II) is sufficient to endow the perfume mixture with an impression of volume, complexity, elegance and/or naturalness,
   and/or
   (iv) the amount of compound of the formula (II) is sufficient, in comparison with an otherwise identically constituted comparative perfume composition without the compound of the formula (II), to create a more beneficial, more harmonic, higher-quality and/or more natural odor impression.

A preferred perfume mixture according to the invention, preferably a perfume oil according to the invention, comprises the compound of the formula (II) in an amount in the range from 0.0001 to 25 wt. %, particularly preferably in the range from 0.001 to 15 wt. %, in each case relative to the total weight of the perfume mixture.

Perfume mixtures according to the invention, in particular perfume oils, preferably comprise two, three, four, five, six, seven, eight, nine, ten or a plurality of perfumes, preferably selected from the substances stated below:

Perfumes that are named in Steffen Arctander, Perfume and Flavor Chemicals, self-published, Montclair, N. J. 1969; H. Surburg, J. Panten, Common Fragrance and Flavor Materials, 5th Edition, Wiley-VCH, Weinheim 2006.

As perfumes that can preferably be combined with the compound according to the invention of the formula (II), we may mention in particular:

Extracts of natural raw materials such as essential oils, concretes, absolutes, resins, resinoids, balsams, tinctures such as e.g. ambergris tincture; amyris oil; angelica seed oil; angelica root oil; anise oil; valerian oil; basil oil; tree moss absolute; bay oil; mugwort oil; benzoin resin; bergamot oil; beeswax absolute; birch tar oil; bitter almond oil; savory oil; buchu leaf oil; cabreuva oil; cade oil; calamus oil; camphor oil; cananga oil; cardamom oil; cascarilla oil; cassia oil; cassie absolute; castoreum absolute; cedar leaf oil; cedar wood oil; cistus oil; citronella oil; lemon oil; copaiba balsam; copaiba balsam oil; coriander oil; costus root oil; cumin oil; cypress oil; davana oil; dill oil; dill seed oil; eau de brouts absolute; oak moss absolute; elemi oil; tarragon oil; eucalyptus citriodora oil; eucalyptus oil; fennel oil; fir-needle oil; galbanum oil; galbanum resin; geranium oil; grapefruit oil; guaiacwood oil; gurjun balsam; gurjun balsam oil, helichrysum absolute; helichrysum oil; ginger oil; iris root absolute; iris root oil; jasmine absolute; calamus oil; chamomile oil blue; Roman chamomile oil; carrot seed oil; cascarilla oil; pine needle oil; spearmint oil; caraway oil; labdanum oil; labdanum absolute; labdanum resin; lavandin absolute; lavandin oil; lavender absolute; lavender oil; lemongrass oil; lovage oil; sweet lime oil distilled; sweet lime oil pressed; linaloe oil; litsea cubeba oil; bay leaf oil; mace oil; marjoram oil; mandarin oil; massoia bark oil; mimosa absolute; musk seed oil; musk tincture; muscatel-sage oil; nutmeg butter; myrrh absolute; myrrh oil; myrtle oil; clove leaf oil; clove blossom oil; neroli oil; olibanum absolute; olibanum oil; opopanax oil; orange blossom absolute; orange oil; origanum oil; palmarosa oil; patchouli oil; perilla oil; Peruvian balsam oil; parsley leaf oil; parsley seed oil; petitgrain oil; peppermint oil; pepper oil; pimenta oil; pine oil; poley oil; rose absolute; rosewood oil; rose oil; rosemary oil; Dalmatian sage oil; Spanish sage oil; sandalwood oil; celery seed oil; spike lavender oil; star anise oil; styrax oil; tagetes oil; fir-needle oil; tea tree oil; turpentine oil; thyme oil; tolu balsam; tonka absolute; tuberose absolute; vanilla extract; violet leaf absolute; verbena oil; vetiver oil; juniper berry oil; wine lees oil; wormwood oil; wintergreen oil; ylang-ylang oil; hyssop oil; civet absolute; cinnamon leaf oil; cinnamon bark oil; and fractions thereof or ingredients isolated therefrom;
individual perfumes from the hydrocarbons group, e.g. 3-carene; α-pinene; β-pinene; α-terpinene; γ-terpinene; p-cymene; bisabolene; camphene; caryophyllene; cedrene; farnesene; limonene; longifolene; myrcene; ocimene; valencene; (E,Z)-1,3,5-undecatriene;
from the group of the aliphatic alcohols, e.g. hexanol; octanol; 3-octanol; 2,6-dimethyl heptanol; 2-methyl heptanol, 2-methyl octanol; (E)-2-hexenol; (E)- and (Z)-3-hexenol; 1-octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;
from the group of the aliphatic aldehydes and acetals thereof, e.g. hexanal; heptanal; octanal; nonanal; decanal; undecanal; dodecanal; tridecanal; 2-methyl octanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-5,9-undecadienal; heptanal-diethylacetal; 1,1-dimethoxy-2,2,5-trimethyl-4-hexene; citronellyloxyacetaldehyde;
from the group of the aliphatic ketones and oximes thereof, e.g. 2-heptanone; 2-octanone; 3-octanone; 2-nonanone; 5-methyl-3-heptanone; 5-methyl-3-heptanone oxime; 2,4,4,7-tetramethyl-6-octen-3-one;
from the group of the aliphatic sulfur-containing compounds, e.g. 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;
from the group of the aliphatic nitriles, e.g. 2-nonenoic acid nitrile; 2-tridecenoic acid nitrile; 2,12-tridecenoic acid nitrile; 3,7-dimethyl-2,6-octadienoic acid nitrile; 3,7-dimethyl-6-octenoic acid nitrile;
from the group of the aliphatic carboxylic acids and esters thereof, e.g. (E)- and (Z)-3-hexenylformate; ethyl acetoacetate; isoamyl acetate; hexyl acetate; 3,5,5-trimethyl hexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate; isoamyl butyrate; hexyl butyrate; (E)- and (Z)-3-hexenyl isobutyrate; hexyl crotonate; ethyl isovalerate; ethyl-2-methylpentanoate; ethyl hexanoate; allyl hexanoate; ethyl heptanoate; allyl heptanoate; ethyl octanoate; ethyl-(E,Z)-2,4-decadienoate; methyl-2-octinate; methyl-2-noninate; allyl-2-isoamyloxyacetate; methyl-3,7-dimethyl-2,6-octadienoate;
from the group of the acyclic terpene alcohols, e.g. citronellol; geraniol; nerol; linalool; lavadulol; nerolidol; farnesol; tetrahydrolinalool; tetrahydrogeraniol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and their formates, acetates, propionates, isobutyrates, butyrates, isovalerates, pentanoates, hexanoates, crotonates, tiglinates, 3-methyl-2-butenoates;
from the group of the acyclic terpene aldehydes and terpene ketones, e.g. geranial; neral; citronellal; 7-hydroxy-3,7-dimethyloctanal; 7-methoxy-3,7-dimethyloctanal; 2,6,10-trimethyl-9-undecenal; geranyl acetone; and the dimethyl and diethyl acetals of geranial, neral, 7-hydroxy-3,7-dimethyloctanal;
from the group of the cyclic terpene alcohols, e.g. menthol; isopulegol; α-terpineol; terpinenol-4; menthan-8-ol; menthan-1-ol; menthan-7-ol; borneol; isoborneol; linalool oxide; nopol; cedrol; ambrinol; vetiverol; guaiol; and their formates, acetates, propionates, isobutyrates, butyrates, isovalerates, pentanoates, hexanoates, crotonates, tiglinates, 3-methyl-2-butenoates;
from the group of the cyclic terpene aldehydes and terpene ketones, e.g. menthone; isomenthone; 8-mercaptomenthan-3-one; carvone; camphor; fenchone; α-ionone; β-ionone; α-n-methylionone; β-n-methylionone; α-isomethylionone; β-isomethylionone; α-irone; α-damascone; β-damascone; β-damascenone; γ-damascone; δ-damascone; 1-(2,4,4-trimethyl-2-cyclohexen-1-yl)-2-buten-1-one; 1,3,4,6,7,8a-hexahydro-1,1,5,5-tetramethyl-2H-2,4-a-methanonaphthalen-8(5H)-one; nootkatone; dihydronootkatone; α-sinensal; β-sinensal; acetylated cedar wood oil (methyl cedryl ketone);
from the group of the cyclic alcohols, e.g. 4-tert-butylcyclohexanol; 3,3,5-trimethylcyclohexanol; 3-isocamphylcyclohexanol; 2,6,9-trimethyl-(Z2,Z5,E9)-cyclododecatrien-1-ol; 2-isobutyl-4-methyltetrahydro-2H-pyran-4-ol;
from the group of the cycloaliphatic alcohols, e.g. α,3,3-trimethylcyclohexylmethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trimethyl-3-cyclopent-1-yl)-2-buten-1-ol; 3-methyl-5-(2,2,3-trimethyl-3-cyclopent-1-yl)-pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;
from the group of the cyclic and cycloaliphatic ethers, e.g. cineol; cedryl methyl ether; cyclododecylmethyl ether; (ethoxymethoxy)cyclododecane; α-cedrene-epoxide; 3a,6,6,9a-tetramethyldodecahydronaphtho[2,1-b]furan; 3a-ethyl-6,6,9a-trimethyldodecahydronaphtho[2,1-b]furan; 1,5,9-trimethyl-13-oxabicyclo[10.1.0]trideca-4,8-diene; rose oxide; 2-(2,4-dimethyl-3-cyclohexen-1-yl)-5-methyl-5-(1-methylpropyl)-1,3-dioxane;
from the group of the cyclic ketones, e.g. 4-tert-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert-pentylcyclohexanone; 5-cyclohexadecen-1-one; 6,7-dihydro-1,1,2,3,3-pentamethyl-4(5H)-indanone; 9-cycloheptadecen-1-one; cyclopentadecanone; cyclohexadecanone;
from the group of the cycloaliphatic aldehydes, e.g. 2,4-dimethyl-3-cyclohexenecarbaldehyde; 2-methyl-4-(2,2,6-trimethyl-cyclohexen-1-yl)-2-butenal; 4-(4-hydroxy-4-methylpentyl)-3-cyclohexenecarbaldehyde; 4-(4-methyl-3-penten-1-yl)-3-cyclohexenecarbaldehyde;
from the group of the cycloaliphatic ketones, e.g. 1-(3,3-dimethylcyclohexyl)-4-penten-1-one; 1-(5,5-dimethyl-1-cyclohexen-1-yl)-4-penten-1-one; 2,3,8,8-tetramethyl-1,2,3,4,5,6,7,8-octahydro-2-naphthalenylmethyl ketone; methyl-2,6,10-trimethyl-2,5,9-cyclododecatrienyl ketone; tert-butyl-(2,4-dimethyl-3-cyclohexen-1-yl)ketone;
from the group of the esters of cyclic alcohols, e.g. 2-tert-butylcyclohexyl acetate; 4-tert-butylcyclohexyl acetate; 2-tert-pentylcyclohexyl acetate; 4-tert-pentylcyclohexyl acetate; decahydro-2-naphthyl acetate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5- or -6-indenyl acetate; 4,7-methano-3a,4,5,6,7,7a-hexahydro-5- or -6-indenylpropionate; 4,7-methano-3a,4,5,6,7,7a-hexahydro-5- or -6-indenylisobutyrate; 4,7-methanooctahydro-5- or -6-indenyl acetate;
from the group of the esters of cycloaliphatic carboxylic acids, e.g. allyl-3-cyclohexylpropionate; allylcyclohexyloxyacetate; methyldihydrojasmonate; methyljasmonate; methyl-2-hexyl-3-oxocyclopentane carboxylate; ethyl-2-ethyl-6,6-dimethyl-2-cyclohexenecarboxylate; ethyl-2,3,6,6-tetramethyl-2-cyclohexenecarboxylate; ethyl-2-methyl-1,3-dioxolan-2-acetate;
from the group of the aromatic hydrocarbons, e.g. styrene and diphenylmethane;
from the group of the araliphatic alcohols, e.g. benzyl alcohol; 1-phenylethyl alcohol; 2-phenylethyl alcohol; 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;
from the group of the esters of araliphatic alcohols and aliphatic carboxylic acids, e.g. benzyl acetate; benzyl propionate; benzyl isobutyrate; benzyl isovalerate; 2-phenylethyl acetate; 2-phenylethylpropionate; 2-phenylethyl isobutyrate; 2-phenylethyl isovalerate; 1-phenylethyl acetate; α-trichloromethylbenzyl acetate; α,α-dimethylphenylethyl acetate; α,α-dimethylphenylethyl butyrate; cinnamyl acetate; 2-phenoxyethyl isobutyrate; 4-methoxybenzyl acetate;
from the group of the araliphatic ethers, e.g. 2-phenylethylmethyl ether; 2-phenylethyl isoamyl ether; 2-phenylethyl-1-ethoxyethyl ether; phenylacetaldehyde-dimethylacetal; phenylacetaldehyde-diethylacetal; hydratropic aldehyde-dimethylacetal; phenylacetaldehyde-glycerinacetal; 2,4,6-trimethyl-4-phenyl-1,3-dioxanes; 4,4a,5,9b-tetrahydroindeno[1,2-d]-dioxin; 4,4a,5,9b-tetrahydro-2,4-dimethylindeno[1,2-d]-m-dioxin;
from the group of the aromatic and araliphatic aldehydes, e.g. benzaldehyde; phenylacetaldehyde; 3-phenylpropanal; hydratropic aldehyde; 4-methylbenzaldehyde; 4-methylphenylacetaldehyde; 3-(4-ethylphenyl)-2,2-dimethylpropanal; 2-methyl-3-(4-isopropylphenyl)propanal; 2-methyl-3-(4-tert-butylphenyl)propanal; 3-(4-tert-butylphenyl)propanal; cinnamaldehyde; α-butylcinnamaldehyde; α-amylcinnamaldehyde; α-hexylcinnamaldehyde; 3-methyl-5-phenylpentanal; 4-methoxybenzaldehyde; 4-hydroxy-3-methoxybenzaldehyde; 4-hydroxy-3-ethoxybenzaldehyde; 3,4-methylene dioxybenzaldehyde; 3,4-dimethoxybenzaldehyde; 2-methyl-3-(4-methoxyphenyl)propanal; 2-methyl-3-(4-methylene dioxyphenyl)propanal;
from the group of the aromatic and araliphatic ketones, e.g. acetophenone; 4-methylacetophenone; 4-methoxyacetophenone; 4-tert-butyl-2,6-dimethylacetophenone; 4-phenyl-2-butanone; 4-(4-hydroxyphenyl)-2-butanone; 1-(2-naphthalenyl)ethanone; benzophenone; 1,1,2,3,3,6-hexamethyl-5-indanylmethyl ketone; 6-tert-butyl-1,1-dimethyl-4-indanylmethyl ketone; 1-[2,3-dihydro-1,1,2,6-tetramethyl-3-(1-methylethyl)-1H-5-indenyl]ethanone; 5′,6′,7′,8′-tetrahydro-3′,5′,5′,6′,8′,8′-hexamethyl-2-acetonaphthone;
from the group of the aromatic and araliphatic carboxylic acids and esters thereof, e.g. benzoic acid; phenylacetic acid; methyl benzoate; ethyl benzoate; hexyl benzoate; benzyl benzoate; methylphenyl acetate; ethylphenyl acetate; geranylphenyl acetate; phenylethyl-phenyl acetate; methyl cinnamate; ethyl cinnamate; benzyl cinnamate; phenylethyl cinnamate; cinnamyl cinnamate; allylphenoxyacetate; methyl salicylate; isoamyl salicylate; hexyl salicylate; cyclohexyl salicylate; cis-3-hexenyl salicylate; benzyl salicylate; phenylethyl salicylate; methyl-2,4-dihydroxy-3,6-dimethyl benzoate; ethyl-3-phenylglycidate; ethyl-3-methyl-3-phenylglycidate;
from the group of the nitrogen-containing aromatic compounds, e.g., 2,4,6-trinitro-1,3-dimethyl-5-tert-butylbenzene; 3,5-dinitro-2,6-dimethyl-4-tert-butylacetophenone; cinnamic acid nitrile; 5-phenyl-3-methyl-2-pentenoic acid nitrile; 5-phenyl-3-methylpentanoic acid nitrile; methylanthranilate; methyl-N-methylanthranilate; Schiff's bases of methylanthranilate with 7-hydroxy-3,7-dimethyloctanal, 2-methyl-3-(4-tert-butylphenyl)propanal or 2,4-dimethyl-3-cyclohexenecarbaldehyde; 6-isopropylquinoline; 6-isobutylquinoline; 6-sec-butylquinoline; indole; skatole; 2-methoxy-3-isopropylpyrazine; 2-isobutyl-3-methoxypyrazine; 4-(4,8-dimethyl-3,7-nonadienyl)-pyridine;
from the group of the phenols, phenyl ethers and phenyl esters, e.g. estragole; anethole; eugenol; eugenyl methyl ether; isoeugenol; isoeugenylmethyl ether; thymol; carvacrol; diphenyl ether; β-naphthylmethyl ether; β-naphthylethyl ether; β-naphthyl isobutyl ether; 1,4-dimethoxybenzene; eugenyl acetate; 2-methoxy-4-methylphenol; 2-ethoxy-5-(1-propenyl)phenol; p-cresylphenyl acetate;
from the group of the heterocyclic compounds, e.g. 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;
from the group of the lactones, e.g. 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 1,15-pentadecanolide; cis- and trans-11-pentadecen-1,15-olide; cis- and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene-1,12-dodecanedioate; ethylene-1,13-tridecanedioate; coumarin; 2,3-dihydrocoumarin; octahydrocoumarin.

The compound of the formula (II) or a mixture according to the invention (as defined above) affects the sensory properties of perfume mixtures in a variety of ways (in this connection, see also the comparative examples given below).

A perfume mixture according to the invention, preferably a perfume oil according to the invention, is preferred that comprises

(a) one, 2, 3 or a plurality of other woody and/or ambergris perfumes,
and/or
(b) one, 2, 3 or a plurality of musk perfumes.

The musk perfume or perfumes of constituent (b) are perfumes that have a musk odor. Such perfumes are known by a person skilled in the art, as “musk” represents a very important olfactory aspect in perfumery.

According to the invention, the mass ratio of the total amount of musk perfumes, thus of constituent (b), to compound (II), i.e. the total weight ratio of (b):compound (II), is preferably greater than or equal to 10:1, preferably it is 10:1 to 100 000:1, more preferably 50:1 to 100 000:1, and particularly preferably 100:1 to 100 000:1.

Perfume mixtures according to the invention, in particular perfume oils, containing compound (II) or a mixture according to the invention (as defined above) and one or a plurality of musk perfumes, in particular those from Table 1 shown below, for which the mass ratio of the total amount of musk perfumes (b) to compound (II) is greater than or equal to 10:1, are even more preferred.

The compound (II) according to the invention or a mixture according to the invention (as defined above) emphasizes in these preferred or particularly preferred perfume mixtures according to the invention, in particular these perfume oils according to the invention, both the erogenous, masculine and animal odor character (i.e. stress this odor character), which is characteristic of all musk compounds and thereby endows a perfume mixture according to the invention, in particular a perfume oil according to the invention, with a softer, more beneficial (and partly more cosmetic) odor impression, which is therefore ultimately of a higher quality overall.

A perfume mixture according to the invention, preferably a perfume oil according to the invention, is preferred wherein the musk perfume or perfumes of constituent (b) are selected from the group of macrocyclic musk perfumes, the nitro-musk perfumes (aromatic musk perfumes with nitro group(s)), the polycyclic musk perfumes and/or the alicyclic musk perfumes.

Perfume mixtures that are preferred according to the invention, preferably perfume oils, contain two, three or a plurality of different musk perfumes as constituent (b), wherein each of the musk perfumes is preferably selected independently of the other musk perfumes from the group of macrocyclic musk perfumes, the nitro-musk perfumes (aromatic musk perfumes with nitro group(s)), the polycyclic musk perfumes and/or the alicyclic musk perfumes.

Preferably the musk perfume or perfumes are selected in connection with the present invention from the following Table 1:

TABLE 1

| TYPE | Product/Brand name | Name/CAS name |
|---|---|---|
| MACRO | EXALTENON | 4-cyclopentadecen-1-ones (4z)-; 4- |
| | | cyclopentadecen-1-one |
| MACRO | CIVETON | 9-cycloheptadecen-1-one, (9z)- |
| MACRO | CYCLOHEXADECANOLIDE, | oxacycloheptadecan-2-one, |
| | DIHYDROAMBRETTOLIDE | ω-hexadecanolide |
| MACRO | ETHYLENE | 1,4-dioxacyclohexadecane-5,16-dione |
| | DODECANEDIOATE | |
| MACRO | GLOBALIDE ® | oxacyclohexadecen-2-one; 15-pentadec- |
| | | (11/12)-enolide |
| MACRO | ETHYLENE BRASSYLATE | 1,4-dioxacycloheptadecane-5,17-dione |
| MACRO | MUSCON | 3-methyl-cyclopentadecanone |
| MACRO | AMBRETTOLIDE | oxacycloheptadec-10-en-2-one |
| MACRO | MUSCENONE | 3-methyl-cyclopentadecenone |
| MACRO | VELVIONE ®, AMBRETONE | 5-cyclohexadecen-1-one |
| MACRO | AURELIONE ® | 7/8-cyclohexadecen-1-one |
| MACRO | GLOBANONE ® | 8-cyclohexadecen-1-one |
| MACRO | ISOMUSCONE ® | cyclohexadecanone |
| MACRO | EXALTOLIDE, MACROLIDE ® | oxacyclohexadecan-2-one |
| MACRO | COSMONE ® | 3-methyl-(5E/Z)-cyclotetradecen-1-one |
| POLY | TRASEOLIDE ® | 1-[2,3-dihydro-1,1,2,6-tetramethyl-3-(1- |
| | | methylethyl)-1H-inden-5-yl]-ethanone |
| POLY | PHANTOLIDE ® | 1-(2,3-dihydro-1,1,2,3,3,6-hexamethyl-1H- |
| | | inden-5-yl)-ethanone |
| POLY | TONALIDE ® | 1-(5,6,7,8-tetrahydro-3,5,5,6,8,8- |
| | | hexamethyl-2-naphthalenyl)-ethanone |
| POLY | CRYSOLIDE | 1-[6-(1,1-dimethylethyl)-2,3-dihydro-1,1- |
| | | dimethyl-1H-inden-4-yl]-ethanone |
| POLY | CHROMANOLIDE ® | tetradecanoic acid, 1-methylethyl ester; |
| | | cyclopenta[g]-2-benzopyran, 1,3,4,6,7,8- |
| | | hexahydro-4,6,6,7,8,8-hexamethyl- |
| POLY | GALAXOLIDE ® | cyclopenta[g]-2-benzopyran, 1,3,4,6,7,8- |
| | | hexahydro-4,6,6,7,8,8-hexamethyl- |
| ALICYC | HELVETOLIDE ® | 1-propanol, 2-[1-(3,3- |
| | | dimethylcyclohexyl)ethoxy]-2-methyl-, 1- |
| | | propanoate |
| NITRO | MOSKENE | 2,3-dihydro-1,1,3,3,5-pentamethyl-4,6- |
| | | dinitro-1H-inden |
| NITRO | MUSK TIBETENE | 1-(1,1-dimethylethyl)-3,4,5-trimethyl-2,6- |
| | | dinitrobenzene |
| NITRO | ORINOX | 1-[4-(1,1-dimethylethyl)-2,6-dimethylphenyl]- |
| | | ethanone |
| NITRO | MUSK XYLENE | 1-(1,1-dimethylethyl)-3,5-dimethyl-2,4,6- |
| | | trinitrobenzene |
| NITRO | MUSK KETONE | 1-[4-(1,1-dimethylethyl)-2,6-dimethyl-3,5- |
| | | dinitrophenyl]-ethanone |
| NITRO | MUSK ALPHA | 1,3-dibromo-2-methoxy-4-methyl-5- |
| | | nitrobenzene |

MACRO = macrocyclic musk perfumes
NITRO = nitro-musk perfumes
POLY = polycyclic musk perfumes
ALICYC = alicyclic musk perfume

Polycyclic and/or macrocyclic musk perfumes are even more preferred as part of constituent (b), and macrocyclic musk perfumes in particular have proved to be particularly advantageous in the sense of the invention, which in their turn are preferably selected from the group consisting of macrocyclic C₁₄-C₁₈ ketones and macrocyclic C₁₄-C₁₈ lactones. Moreover, ketones or lactones with a ring size of 15 to 17 ring atoms are preferred, which preferably have no, one or two oxygen atoms in the ring.

A perfume mixture according to the invention, preferably a perfume oil according to the invention, with mass ratio of the total amount of musk perfumes of constituent (b) to compound (II) of 10:1 to 100 000:1, even more preferably 50:1 to 100 000:1, particularly preferably 100:1 to 100 000:1, is even more preferred.

Perfume mixtures according to the invention are most preferred whose constituent (b) comprises or is selected from: 3-methylcyclopentadecenone (muscenone), 15-pentadec-(11/12)-enolide (Globalide)®, ethylene brassylate, oxacyclohexadecan-2-one (Macrolide®), cyclohexadecanone (Isomuscone®), 8-cyclohexadecanone (Globanone®), (7/8)-cyclohexadecanone (Aurelione®) and mixtures thereof.

In a preferred embodiment, constituent (b) is selected from the group consisting of 15-pentadec-(11/12)-enolide (Globalide®), ethylene brassylate and oxacyclohexadecan-2-one (Macrolide®) and mixtures thereof.

The animal character of the musk perfumes is harmonized and rounded by adding the compound of the formula (II) or a mixture according to the invention (as defined above) in particular with the preferably one, two, three, four, five, six, seven, eight, nine, ten or a plurality of further perfumes, which are preferably a constituent of a perfume mixture according to the invention, preferably a preferred perfume oil according to the invention. This results in a softer, more beneficial and thus ultimately a higher-quality overall impression.

As already mentioned, the compound according to the invention of the formula (II) is, on account of its olfactory properties, particularly suitable for use in perfume mixtures and perfume oils. The compound of the formula (II) can be used alone or in mixtures according to the invention together with the compound of the formula (I) in corresponding perfume mixtures together with another individual perfume or with a large number of other perfumes. Especially advantageously, the compound of formula (II) can be combined with other perfumes, preferably selected from the perfumes already mentioned above or stated below, in various proportions, to novel perfume mixtures or perfumes.

Furthermore, additional olfactory effects were found.

The invention further relates to methods of intensifying an odor with one of the notes fruity, flowery, spicy, woody, musk and/or ambergris-like, comprising the following step:
mixing of
   one or a plurality of perfumes with one, a plurality of or all of the notes fruity, flowery, spicy, woody, musk and/or ambergris-like,
   (i) with an amount of compound (II) according to the invention or a mixture according to the invention (as defined above), that is sufficient to emphasize the odor impression of the perfume or perfumes, which produce one, a plurality of or all of the notes fruity, flowery, spicy, woody, musk and/or ambergris-like,
   or
   (ii) with a perfume mixture according to the invention, preferably a perfume oil according to the invention, preferably in one of the embodiments characterized as preferable, in an amount that is sufficient to emphasize the odor impression of the perfume or perfumes that produce one, a plurality of or all of the notes fruity, flowery, spicy, woody, musk and/or ambergris-like.

The compound according to the invention of the formula (II), mixtures according to the invention or perfume mixtures according to the invention are preferably used in the production of perfumed products (perfumed articles). The sensory properties as well as the material properties (such as solubility in common solvents and compatibility with common further constituents of such products) and the toxicological harmlessness of the compound according to the invention underscore their particular suitability for the stated applications.

Accordingly, within the scope of the present invention, a perfumed product is provided, which contains the compound of the formula (II), a mixture according to the invention as defined above or a perfume mixture according to the invention as defined above. With respect to preferred embodiments, the statements made above apply correspondingly.

Preferred perfumed products according to the invention are selected from the group consisting of:

Perfume extracts, eau de parfum, eau de toilette, aftershave, eau de cologne, pre-shave products, splash-cologne, perfumed tissue wipes, perfumes for acidic, alkaline and neutral cleansing agents, detergents, washing tablets, disinfectants, as well as air fresheners, aerosol sprays, waxes and polishes, and body care products, bath oils, cosmetic emulsions, e.g. skin creams and lotions, sunscreen creams and lotions, after-sun creams and lotions, hand creams and lotions, foot creams and lotions, depilatory creams and lotions, after-shave creams and lotions, tanning creams and lotions, hair care products e.g. hair sprays, hair gels, hair fixing lotions, hair rinses, hair coloring agents, hair shaping agents and hair straighteners, hair lotions, hair creams and lotions, deodorants and antiperspirants, products of decorative cosmetics e.g. eye shadows, nail varnishes, make-up, lipsticks, mascara as well as candles, lamp oils, joss sticks, insecticides, repellents, propellants.

Perfume mixtures according to the invention containing the compound of formula (II) or a mixture according to the invention as defined above can generally be used (e.g. in concentrated form, in solutions or in modified form described below) for the production of e.g. perfume extracts, eau de parfum, eau de toilette, aftershave, eau de cologne, pre-shave products, splash-cologne and perfumed tissue wipes and the perfuming of acidic, alkaline and neutral cleaning agents, e.g. floor cleaners, window cleaners, washing-up liquids, bath and sanitary cleaners, liquid scouring cleaners, solid and liquid lavatory cleaners, powder and foam carpet cleaners, liquid detergents, powder detergents, clothes-washing pre-treatment agents such as bleaching agents, soaking agents and stain-removers, clothes-washing rinses, washing soaps, washing tablets, disinfectants, surface disinfectants and air fresheners in liquid form, gel form or applied on a solid carrier, aerosol sprays, waxes and polishes such as furniture polishes, floor polishes, shoe creams and body care products, for example solid and liquid soaps, shower gels, shampoos, shaving soaps, shaving foams, bath oils, cosmetic emulsions of the oil-in-water, water-in-oil and water-in-oil-in-water type such as for example skin creams and lotions, face creams and lotions, sunscreen creams and lotions, after-sun creams and lotions, hand creams and lotions, foot creams and lotions, depilatory creams and lotions, after-shave creams and lotions, tanning creams and lotions, hair care products e.g. hair sprays, hair gels, hair fixing lotions, hair rinses, permanent and semi-permanent hair coloring agents, hair shaping agents such as cold waving agents and hair straighteners, hair lotions, hair creams and lotions, deodorants and antiperspirants, for example underarm sprays, roll-ons, stick deodorants, deodorant creams, products of decorative cosmetics, for example eye shadows, nail varnishes, make-up, lipsticks, mascara, as well as candles, lamp oils, joss sticks, insecticides, repellents, propellants.

A perfumed product according to the invention contains compound (II), a mixture according to the invention (as defined above) or a perfume mixture (as defined above), preferably in a sensorially effective amount.

The foregoing applies correspondingly to the compound of the formula (II) contained in perfumed products according to the invention, a mixture according to the invention as defined above or a perfume mixture according to the invention as defined above and the preferred embodiments thereof.

The aforementioned perfume mixtures according to the invention or the perfume mixtures to be used according to the invention in the corresponding products can be used for perfuming in liquid form, undiluted or diluted with a solvent. Suitable solvents for this are e.g. ethanol, isopropanol, diethylene glycol monoethyl ether, glycerol, propylene glycol, 1,2-butylene glycol, dipropylene glycol, diethyl phthalate, triethyl citrate, isopropyl myristate etc. Within the context of the present text, in the case when the aforementioned solvents have intrinsic olfactory properties, it is to be noted that they are to be assigned exclusively to the constituent “solvent” and not to the “perfumes”.

The compound of the formula (II) contained in the perfumed products according to the invention, a mixture according to the invention as defined above or a perfume mixture according to the invention as defined above can, in a preferred embodiment, be absorbed on a carrier substance, which provides both fine distribution of the perfumes in the product and controlled release during use. Said carriers can be porous inorganic materials such as light sulfate, silica gels, zeolites, gypsums, clays, clay granules, porous concrete etc. or organic materials such as wood and cellulose-based materials.

The compound of the formula (II) contained in the perfumed products according to the invention, a mixture according to the invention as defined above or a perfume mixture according to the invention as defined above can also be in the form of microencapsulated, spray-dried, inclusion complexes or extrusion products and can be added in this form to the product, or article, to be perfumed.

Optionally the properties of the perfume mixtures modified in this way can be further optimized by so-called “coating” with suitable materials with respect to more-targeted fragrance release, for which preferably wax-like plastics, e.g. polyvinyl alcohol are used.

The microencapsulation of the perfume mixtures can be carried out for example by the so-called coacervation method using capsule materials, e.g. of polyurethane-like substances or soft gelatins. The spray-dried perfume oils can for example be produced by spray-drying of an emulsion or dispersion containing the perfume oil, using modified starch, proteins, dextrin and plant gums as carrier substances. Inclusion complexes can be prepared e.g. by introducing dispersions of the perfume mixture and cyclodextrins or urea derivatives into a suitable solvent, e.g. water. Extrusion products can be prepared by melting the perfume mixture with a suitable wax-like substance and by extrusion with subsequent solidification, optionally in a suitable solvent, e.g. isopropanol.

The perfume mixtures according to the invention can accordingly, as already mentioned, be used in concentrated form, in solutions or in the modified form described above for the production of the corresponding perfumed articles according to the invention.

Ingredients with which the compound according to the invention of the formula (II), a mixture according to the invention as defined above or a perfume mixture according to the invention as defined above, can preferably be combined, are for example: preservatives, abrasives, anti-acne agents, agents against skin ageing, antibacterial agents, anticellulitis agents, antidandruff agents, anti-inflammatory agents, agents for preventing irritation, agents inhibiting irritation, antimicrobial agents, antioxidants, astringents, sweat inhibiting agents, antiseptic agents, antistatic agents, binders, buffers, carrier materials, chelating agents, cell stimulants, cleaning agents, care agents, depilatory agents, surface-active substances, deodorants, antiperspirants, plasticizers, emulsifiers, enzymes, essential oils, fibers, film-forming agents, fixatives, foaming agents, foam stabilizers, substances to prevent foaming, foam boosters, fungicides, gelling agents, gel-forming agents, hair care agents, hair shaping agents, hair straighteners, hydrating agents, moisturizers, humectants, bleaching agents, starching agents, stain-removing agents, optical brighteners, impregnating agents, anti-soiling agents, friction-reducing agents, lubricants, moisturizing creams, ointments, opacifiers, plasticizing agents, covering agents, polish, brighteners, polymers, powders, proteins, refatting agents, abrading agents, silicones, skin calmatives, skin cleansing agents, skin care agents, skin healing agents, skin clarifying agents, skin protecting agents, skin softening agents, cooling agents, skin cooling agents, warming agents, skin warming agents, stabilizers, UV-absorbing agents, UV filters, detergents, fabric softeners, suspending agents, tanning agents, thickeners, vitamins, oils, waxes, fats, phospholipids, saturated fatty acids, mono- or poly-unsaturated fatty acids, α*hydroxy acids, polyhydroxy fatty acids, liquefiers, dyes, color-protecting agents, pigments, anticorrosive agents, aromas, flavoring agents, polyols, surfactants, electrolytes, organic solvents or silicone derivatives.

In the following, further aspects according to the invention are described in connection with the compound according to the invention of the formula (II), a mixture according to the invention (as defined above) or a perfume mixture according to the invention (as defined above).

As already explained, for surfactant-containing perfumed products the substantivity of a perfume or of a perfume mixture with respect to the substrate or its retention on the substrate, in particular hair or textile fibers, is another important application requirement.

By adding the compound according to the invention of the formula (II) or a mixture according to the invention (as defined above) to a given perfume mixture of only slight substantivity and/or retention, these properties are improved particularly advantageously. Thus, for example an aqueous washing solution (or a corresponding detergent or shampoo or the like) that does indeed have a fruity, flowery, and/or spicy fragrance, but because of inadequate substantivity of the odorants it contains, is unsuitable for imparting a fruity, flowery and/or spicy odor to laundry (textile fibers) or hair, can, by adding the compound according to the invention of the formula (II) or a mixture according to the invention (as defined above) be transformed into a solution that is excellent for imparting a fruity, flowery and/or spicy odor—and the fruity, flowery and/or spicy odor persists for a long time on the treated substrates (hair or textile fibers).

The compound according to the invention of the formula (II) and/or a mixture according to the invention (as defined above) containing perfume mixtures (as characterized above) are characterized by high substantivity (as defined above). This effect is particularly noticeable on substrates such as skin, hair and textile fibers (e.g. wool, cotton, linen, synthetic fibers).

This effect is discussed in more detail below within the context of the application examples.

A product perfumed according to the invention is therefore wherein the product contains one or a plurality of surfactants.

It is preferable if the product is one of the following:

a weakly acid, alkaline or neutral cleaning agent, selected in particular from the group consisting of
all-purpose cleaners, floor cleaners, window glass cleaners, washing-up liquids, bath and sanitary cleaners, liquid scouring cleaners, solid and liquid lavatory cleaners, powder and foam carpet cleaners, liquid detergents, powder detergents, clothes-washing pre-treatment agents such as bleach, soaking agents and stain-removers, clothes-washing rinses, washing soaps, washing tablets, disinfectants, surface disinfectants,
air fresheners in the form of liquid or gel or applied on a solid carrier or as aerosol spray,
waxes or polishes, selected in particular from the group consisting of furniture polishes, floor polishes and shoe creams,
   or
body care products, selected in particular from the group consisting of shower gels and shampoos.

The positive properties described mean that the compound according to the invention of the formula (II), a mixture according to the invention (as defined above) or a perfume mixture according to the invention (as defined above) can be used particularly preferably in washing and cleaning agents, hygiene or care products, in particular in the area of body and hair care, cosmetics and household products.

Particularly preferred perfumed products according to the invention are therefore washing and cleaning agents, hygiene or care products, in particular in the area of body and hair care, cosmetics and household products.

A perfume mixture according to the invention is prepared according to the invention by mixing the compound according to the invention of formula (II) or a mixture according to the invention as defined above with the further perfume(s) and optionally further constituents of the perfume mixture.

According to a preferred embodiment, a perfume mixture according to the invention as described above is prepared, wherein the compound according to the invention of the formula (II) is used in an amount that is sufficient to impart, to modify and/or to intensify one or preferably both of the odor notes ambergris and wood in the perfume mixture.

The foregoing applies correspondingly to the further constituents or perfumes that are preferably to be selected in addition to the compound according to the invention of formula (II).

As already mentioned, the compound according to the invention of the formula (II) can be used as a means of providing hair and/or textile fibers with one or both of the odor notes ambergris and/or wood.

Accordingly, a further aspect of the present invention relates to a method of providing (a) hair or (b) textile fibers with one or both of the odor notes ambergris and/or wood, comprising the following steps:
i) preparing the compound of the formula (II) or a mixture according to the invention (as defined above),
ii) applying the compound or the mixture according to the invention on (a) the hair or (b) the textile fibers.

The invention further relates to a method of providing (a) hair or (b) textile fibers with one, a plurality of or all of the odor notes fruity, flowery, spicy, woody, musk and/or ambergris-like with the following steps:
preparing a perfume mixture according to the invention, preferably in one of the embodiments characterized as preferable, comprising
   (a) compound (II) or a mixture according to the invention (preferably in one of the embodiments characterized as preferable),
   and
   (b) one or a plurality of further perfumes, which impart one, a plurality of or all of the notes fruity, flowery, spicy, woody, musk and/or ambergris-like, wherein the amount of compound (II) is sufficient to modify and/or intensify one or a plurality of these notes,
applying the mixture on (a) the hair or (b) the textile fibers.

### EXAMPLES

The following examples explain the invention; unless stated otherwise, proportions and percentages are based on weight.

### Abbreviations Used:

Dipropylene glycol (DPG), diethyl phthalate (DEP), triethyl citrate (TEC).

For explanations of the product names of the perfumes, see e.g. S. Arctander, Perfume and Flavor Materials, Vol. I and II, Montclair, N. J., 1969, self-published or H. Surburg, J. Panten, “Common Fragrance and Flavor Materials”, 5^{th}. Ed., Wiley-VCH, Weinheim 2006.

cis-Cedranediol was used, and was obtained by epoxidation and subsequent ring opening from (−)-α-cedrene. According to NMR analysis the diol has the following structure:

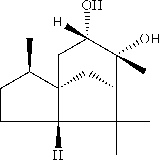

### Example 1

### Reaction of cis-Cedranediol with Pentan-2-One

24 g (0.1 mol) of cis-cedranediol (26 g with a GC content of 92% was used), 125 g (1.45 mol) of methyl-n-propyl-ketone, 31.8 g (0.3 mol) of orthoformic acid trimethyl ester and 0.4 g of concentrated sulfuric acid were stirred at 10° C. for 24 hours. At the end of reaction, methyl-tert.-butyl ether was added, it was washed with a 5% sodium hydrogen carbonate solution and the low-boiling components were distilled off. After adding n-hexane, unreacted cis-cedranediol crystallized out, and was separated by filtration. After concentrating the filtrate by evaporation, 30.4 g of raw product was obtained. According to GC analysis, the total content of the compounds of formulae (I) and (II) in the product obtained was 51% (corresponding to a yield of 50% of theory). By dissolving the raw product in dipropylene glycol (DPG), a solution was prepared with a content of 10 wt. % of compounds of formula (I) and (II) (with a ratio of (I):(II)=4:3).

For analytical investigations, the stereoisomeric compounds of formula (I) and (II) from the raw product were separated from one another by liquid chromatography on silica gel 60 (0.04-0.063 mm) with the eluent cyclohexane/ethyl acetate (60/1). The compound of formula (I) was obtained with a purity of 99.4% and the compound of the formula (II) with a purity of 96.9%.

### Compound of the Formula (I):

1H-NMR data (ppm): 4.06 d, d 9.0 Hz, 6.7 Hz 1H; 2.01 d 12.1 Hz 1H; 1.93 d 4.5 Hz 1H; 1.91 d, d, d 13.5 Hz, 9.1 Hz, 2.5 Hz 1H; 1.84 m 1H; 1.80-1.70 m 3H; 1.65-1.37 m 7H; 1.50 s 3H; 1.45 s 3H; 1.34-1.24 m 1H; 1.16 s 3H; 1.04 s 3H; 0.94 t 7.4 Hz 3H; 0.82 d 7.1 Hz 3H.

13C-NMR data (ppm): 110.6 s; 84.7 d; 78.6 s; 58.9 d; 57.4 d; 52.3 s; 45.0 t; 42.4 s; 42.0 d; 41.1 t; 38.5 t; 36.0 t; 31.2 q; 28.8 q; 27.8 q; 27.2 q; 25.5 t; 18.3 t; 15.4 q; 14.7 q.

Mass spectrum: 306 (1.8), 263 (30), 203 (36), 133 (31), 119 (59), 105 (30), 91 (18), 69 (40), 55 (32), 43 (100)

Odor: faint ambergris

### Compound of the Formula (II):

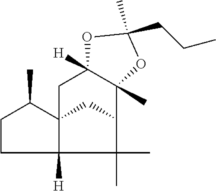

1H-NMR data (ppm): 4.05 d, d 9.0 Hz, 6.7 Hz, 1H; 2.00 d 12.1 Hz 1H; 1.94 d 4.5 Hz 1H; 1.94 d, d, d 13.4 Hz, 9.1 Hz, 2.5 Hz 1H; 1.88-1.50 m 8H; 1.49 s 3H; 1.48 s 3H; 1.49-1.40 m 3H; 1.34-1.24 m 1H; 1.16 s 3H; 1.04 s 3H; 0.93 t 7.4 Hz 3H; 0.82 d 7.1 Hz 3H.

13C-NMR data (ppm): 110.7 s; 84.9 d; 78.5 s; 58.7 d; 57.5 d; 52.4 s; 45.9 t; 42.4 s; 41.9 d; 41.3 t; 38.5 t; 35.8 t; 31.2 q; 28.7 q; 27.8 q; 26.7 q; 25.4 t; 18.5 t; 15.4 q; 14.6 q.

Mass spectrum: 306 (1.2), 263 (36), 203 (46), 133 (40), 119 (68), 105 (34), 91 (19), 69 (44), 55 (32), 43 (100)

Odor: very strong ambergris, wood, reminiscent of white amber, very persistent

### Comparative Example

### Reaction of cis-Cedranediol with Butan-2-One

29.7 g (0.125 mol) of cis-cedranediol (40 g with a GC content of 74.3% was used), 125 g (1.74 mol) of butan-2-one, 40 g (0.375 mol) of orthoformic acid trimethyl ester and 0.5 g concentrated sulfuric acid were stirred at 10° C. for 16 hours. At the end of reaction, methyl-tert.-butyl ether was added, it was washed with a 5% sodium hydrogen carbonate solution and the low-boiling components were distilled off. After adding n-hexane, unreacted cis-cedranediol crystallized out, and was separated by filtration. After concentrating the filtrate by evaporation, 32 g of raw product was obtained. According to GC analysis the total content of the compounds of the formulae (Z-1) and (Z-2) in the product obtained was 42% (corresponding to yield of 37% of theory).

After bulb tube distillation, the compound of the formula (Z-1) was isolated with a purity of 99.8% by liquid chromatography on silica gel 60 (0.04-0.063 mm) with the eluent cyclohexane/methyl-tert.-butyl ether (50/2) and subsequent recrystallization from ethanol. After bulb tube distillation and liquid chromatography on silica gel 60, the compound of formula (Z-2) is additionally obtained by high-performance liquid chromatography at a purity of 99.3%.

### Compound of the Formula (Z-1):

1H-NMR data (ppm): 4.06 d, d 9.0 Hz, 6.8 Hz 1H; 2.00 d 12.2 Hz 1H; 1.94 d 4.5 Hz 1H; 1.92 d, d, d 13.5 Hz, 8.9 Hz, 2.6 Hz 1H; 1.88-1.70 m 4H; 1.66-1.40 m 5H; 1.51 s 3H; 1.44 s 3H; 1.35-1.24 m 1H; 1.16 s 3H; 1.04 s 3H; 1.00 t 7.6 Hz 3H; 0.81 d 7.1 Hz 3H.

13C-NMR data (ppm): 111.1 s; 84.8 s; 78.7 d; 58.6 d; 57.3 d; 52.4 s; 42.5 s; 41.9 d; 41.1 t; 38.5 t; 35.8 t; 35.3 t; 31.2 q; 28.7 q; 27.8 q; 26.5 q; 25.4 t; 15.4 q; 9.3 q

Mass spectrum: 292 (2.3), 263 (37), 221 (100), 203 (49), 133 (48), 119 (88), 105 (45), 69 (55), 55 (45), 43 (87), 41 (51)

Odor: faint ambergris

### Compound of Formula (Z-2):

1H-NMR data (ppm): 4.06 d, d 9.0 Hz, 6.8 Hz 1H; 2.01 d 12.1 Hz 1H; 1.95 d, d, d 13.5 Hz, 9.1 Hz, 2.5 Hz 1H; 1.94 d 4.5 Hz 1H; 1.89-1.40 m 9H; 1.49 s 3H; 1.47 s 3H; 1.34-1.25 m 1H; 1.16s 3H; 1.04 s 3H; 0.98 t 7.6 Hz 3H; 0.82 d 7.1 Hz 3H.

13C-NMR data (ppm): 111.1 s; 84.9 s; 78.6 d; 58.7 d; 57.4 d; 52.4 s; 42.5 s; 41.9 d; 41.3 t; 38.5 t; 36.1 t; 35.8 t; 31.2 q; 28.7 q; 27.8 q; 25.9 q; 25.4 t; 15.4 q; 9.5 q.

Mass spectrum: 292 (1.6), 263 (37), 221 (100), 203 (53), 133 (53), 119 (96), 105 (52), 69 (61), 55 (49), 43 (96), 41 (57)

Odor: ambergris, wood

In the following perfume oil recipes and application examples, solutions with the stated content were used in each case, the weight ratio of the compound of the formula (I) to the compound of the formula (II)) being 4:3.

### Perfume Oil According to the Invention

### Example P1

| | Ref-1 | P1 |
|---|---|---|
| HEXENALDIMETHYLACETAL, 2,2,5-TRIMETHYL-4- | 10 | 10 |
| HEXADECEN-1,16-OLIDE, 7(9)Z- | 15 | 15 |
| CYCLOHEXADECENONE, (8E/Z)- + | 140 | 140 |
| CYCLOHEXADECENONE, (7E)- | | |
| BUT-2-EN-1-ONE, 1-(2,6,6-TRIMETHYL-CYCLOHEX- | 10 | 10 |
| 3-ENYL)-(E) 10% DPG | | |
| ETHANEDIOL BRASSYLATE | 120 | 120 |
| NONADIEN-3-OL, 3,7-DIMETHYL-1,6- | 30 | 30 |
| PYRANOL, 2-ISOBUTYL-4-METHYL-4- | 40 | 40 |
| TETRAHYDRO-(E/Z) | | |
| 5-HEXENYL BUTYRATE, 2-METHYL- | 5 | 5 |
| GERANYL ACETATE | 40 | 40 |
| METHYL-DIHYDROJASMONATE | 400 | 400 |
| 2-(1-(3,3-DIMETHYLCYCLOHEXYL)-ETHOXY-2- | 30 | 30 |
| METHYLPROPYL PROPIONATE | | |
| 3Z-HEXENYL ACETATE | 5 | 5 |
| 3Z-HEXENYL SALICYLATE | 20 | 20 |
| BICYCLO[4.4.0]DECENE, 3,4,10,10-TETRAMETHYL-3- | 50 | 50 |
| HYDROXYETHYL-1 (6) | | |
| CYCLOHEXANONE, 3.3,5,5-TETRAMETHYL-4-(1- | 10 | 10 |
| ETHOXYVINYL)- | | |
| 3Z-HEXENYLMETHYL CARBONATE, 10% in DPG | 10 | 10 |
| MANDARIN OIL | 20 | 20 |
| 2 (3H)-FURANONE, 5-HEXYL-DIHYDRO-4-METHYL- | 5 | 5 |
| (E/Z) | | |
| PRENYL ACETATE | 5 | 5 |
| TRICYCLO(5.2.1.0)DECANE, 8-FORMYL- | 15 | 15 |
| DIOXOL, 4H-4A,9-METHANOAZULENO(5,6-D)- | 20 | 0 |
| OCTAHYDRO-2,2,5,8,8,9A-HEXAMETHYL-, 1,3- | | |
| (Ambrocenide ®), 0.1% in DPG | | |
| Dioxatetracyclo[6.5.1.0^{1,10}.0^{3,7})tetradecane, 5,7,9,9,13- | 0 | 20 |
| pentamethyl-5-propyl-4,6-(compound of formula (II)), 0.1% | | |
| in DPG | | |
| TOTAL | 1000 | 1000 |

The comparative example (Ref-1) with Ambrocenide® smells fresh, fruity and transparent. In perfume oil P1, the compound of formula (II) endows the composition with an elegance and more pronounced fruitiness.

### Perfume Oil According to the Invention

### Example P2

| | Ref-2 | P2 |
|---|---|---|
| UNDECALACTONE, GAMMA- | 2 | 2 |
| HEXADECEN-1,16-OLIDE, 7 (9)Z- | 10 | 10 |
| CYCLOHEXADECENONE, (8E/Z)- + | 100 | 100 |
| CYCLOHEXADECENONE, (7E)- | | |
| CARDAMOM OIL CEYLON | 2 | 2 |
| INDAN-4-ONE, 1,1,2,3,3-PENTAMETHYL- | 15 | 15 |
| TETRAHYDRO- | | |
| CEDAR WOOD OIL VIRGINIA | 100 | 100 |
| DAMASCONE, -BETA-, 10% in DPG | 2 | 2 |
| METHYL-DIHYDROJASMONATE | 210 | 210 |
| BICYCLO[4.4.0]DECENE, 3,4,10,10-TETRAMETHYL-3- | 300 | 300 |
| HYDROXYETHYL-1 (6) | | |
| IRALDEIN, GAMMA- | 60 | 60 |
| CYCLOHEXANONE, 3.3,5,5-TETRAMETHYL-4- | 10 | 10 |
| (1-ETHOXYVINYL)- | | |
| CYCLOPENTADECENONE, 3-METHYL-5E- | 20 | 20 |
| PATCHOULI OIL | 5 | 5 |
| PENTEN-2-OL, 3,3-DIMETHYL-5-(2,2,3-TRIMETHYL- | 20 | 20 |
| 3-CYCLOPENTENYL)-4- | | |
| PENTANOL, 3-METHYL-5-(2,2,3-TRIMETHYL-3- | 80 | 80 |
| CYCLOPENTENYL)-2- + HEXANOL, 6- | | |
| (2,2,3-TRIME-3-CYCLOPENTENYL)-3- | | |
| VANILLIN | 2 | 2 |
| ISOLONGIFOLANONE-ETHANEDIOL KETAL | 30 | 30 |
| CINNAMON BARK OIL CEYLON | 2 | 2 |
| DIOXOL, 4H-4A,9-METHANOAZULENO(5,6-D)- | 30 | 0 |
| OCTAHYDRO-2,2,5,8,8,9A-HEXAMETHYL-, 1,3- | | |
| (Ambrocenide ®), 0.1% in DPG | | |
| Dioxatetracyclo[6.5.1.0^{1,10}.0^{3,7})tetradecane, | 0 | 30 |
| 5,7,9,9,13-pentamethyl-5-propyl-4,6- | | |
| (compound of formula (II)), 0.1% in DPG | | |
| TOTAL | 1000 | 1000 |

The comparative example (Ref-2) with Ambrocenide® has a flowery-spicy odor with dry woody accents. In perfume oil P2 the spicy note is supported by the compound of the formula (II), so that the whole composition appears more peppery and with more character.

### Perfume Oil According to the Invention

### Example P3

| | Ref-3 | P3 |
|---|---|---|
| HEXENALDIMETHYLACETAL, 2,2,5-TRIMETHYL-4- | 10 | 10 |
| BENZODIOXEPINONE, 7-METHYL-3,4-DIHYDRO-3- | 10 | 10 |
| PROPANAL, 2-METHYL-3-(4-METHOXYPHENYL)- | 5 | 5 |
| CITRONELLOL, BETA- | 20 | 20 |
| LEMON OIL | 15 | 15 |
| CYCLOHEXYLOXYALLYL ACETATE, 10% in DPG | 10 | 10 |
| DAMASCONE, TRANS-ALPHA-, 1% in DPG | 20 | 20 |
| BUT-2-EN-1-ONE, 1-(2,6,6-TRIMETHYL- | 5 | 5 |
| CYCLOHEX-3-ENYL)-(E), 10% in DPG | | |
| DECALACTONE GAMMA, 10% in DPG | 15 | 15 |
| ETHANEDIOL BRASSYLATE | 80 | 80 |
| NONADIEN-3-OL, 3,7-DIMETHYL-1,6- | 70 | 70 |
| PHENOL, 2-METHOXY-4-(2-PROPENYL)- | 5 | 5 |
| PYRANOL, 2-ISOBUTYL-4-METHYL-4- | 80 | 80 |
| TETRAHYDRO-(E/Z) | | |
| 5-HEXENYL BUTYRATE, 2-METHYL- | 15 | 15 |
| METHYL-DIHYDROJASMONATE | 250 | 250 |
| PROPANAL, 2-PIPERONYL- | 30 | 30 |
| 3Z-HEXENYL SALICYLATE | 5 | 5 |
| INDOLE, 10% in DPG | 10 | 10 |
| IRALDEIN, GAMMA- | 30 | 30 |
| BICYCLO[4.4.0]DECENE, 3,4,10,10-TETRAMETHYL-3- | 80 | 80 |
| HYDROXYETHYL-1 (6) | | |
| PENTADECANOLIDE, 1,15- | 20 | 20 |
| HEPTENAL, 2,6-DIMETHYL-5-, 10% in DPG | 3 | 3 |
| PYRIDINE, 4-DECYL-, 1% in DPG | 2 | 2 |
| CYCLOHEXENE, 2,4-DIMETHYL-1-FORMYL-3-, | 10 | 10 |
| 10% in DPG | | |
| DIOXOL, 4H-4A,9-METHANOAZULENO(5,6-D)- | 5 | 0 |
| OCTAHYDRO-2,2,5,8,8,9A-HEXAMETHYL-, | | |
| 1,3-(Ambrocenide ®), 0.1% in DPG | | |
| Dioxatetracyclo[6.5.1.0^{1,10}.0^{3,7})tetradecane, | 0 | 5 |
| 5,7,9,9,13-pentamethyl-5-propyl-4,6- | | |
| (compound of formula (II)), 0.1% in DPG | | |
| TOTAL | 805 | 805 |

The comparative example (Ref-3) with Ambrocenide® has impressive transparent freshness. In perfume oil P3, owing to the compound of the formula (II), the composition is perceived as more cosmetic and more beneficial with more volume.

### Perfume Oil According to the Invention

### Example P4

| | Ref-4 | P4 |
|---|---|---|
| NAPHTHO[2.1-B]FURAN, DODECAHYDRO-3A.6.6.9A-TETRAMETHYL- | 5 | 5 |
| BENZOIC SIAM ABS., 50% in DPG | 10 | 10 |
| BENZYL SALICYLATE | 20 | 20 |
| BERGAMOT OIL | 50 | 50 |
| CEDRYLMETHYL ETHER | 40 | 40 |
| CISTUS LABDANUM | 35 | 35 |
| CISTUS OIL SPAN., 10% in DPG | 10 | 10 |
| DAMASCONE, TRANS-ALPHA-, 1% in DPG | 20 | 20 |
| MYRCENOL, DIHYDRO- | 30 | 30 |
| ETHANEDIOL BRASSYLATE | 120 | 120 |
| METHYL BENZOATE, 2,4-DIHYDROXY-3,6-DIMETHYL-, 1% in DPG | 20 | 20 |
| PYRANOL, 2-ISOBUTYL-4-METHYL-4-TETRAHYDRO-(E/Z) | 20 | 20 |
| PENTADECEN-1,15-OLIDE, (11E/Z)- + PENTADECEN-1,15-OLIDE, | 30 | 30 |
| 12E- | | |
| METHYL-DIHYDROJASMONATE | 300 | 300 |
| BICYCLO[4.4.0]DECENE, 3,4,10,10-TETRAMETHYL-3- | 75 | 75 |
| HYDROXYETHYL-1 (6) | | |
| ISOBUTYLQUINOLINE, 10% in DPG | 3 | 3 |
| CYCLOHEXANONE, 3,3,5,5-TETRAMETHYL-4-(1-ETHOXYVINYL)- | 30 | 30 |
| LABDANUM ABSOLUTE | 20 | 20 |
| LINALYL ACETATE | 50 | 50 |
| MANDARIN OIL | 10 | 10 |
| CYCLOPENTADECENONE, 3-METHYL-5E- | 10 | 10 |
| MUSCATEL SAGE OIL | 10 | 10 |
| OLIBANUM COEUR, 50% in TEC | 5 | 5 |
| PATCHOULI OIL | 20 | 20 |
| PYRIDINE, 4-DECYL-, 1% in DPG | 2 | 2 |
| BUTENOL, 2-ETHYL-4-(2,2,3-TRIMETHYL-3-CYCLOPENTENYL)-2E- | 5 | 5 |
| VANILLIN | 30 | 30 |
| DIOXOL, 4H-4A,9-METHANOAZULENO(5,6-D)-OCTAHYDRO- | 20 | 0 |
| 2,2,5,8,8,9A-HEXAMETHYL-, 1,3-(Ambrocenide ®), 0.1% in DPG | | |
| Dioxatetracyclo[6.5.1.0^{1,10}.0^{3,7})tetradecane, 5,7,9,9,13-pentamethyl-5- | 0 | 20 |
| propyl-4,6-(compound of formula (II)), 0.1% in DPG | | |
| TOTAL | 1000 | 1000 |

The comparative example (Ref-4) with Ambrocenide® is impressive with a fine woody note. In perfume oil P4, the compound of the formula (II) endows the composition with more volume and the tobacco note is intensified.

### Perfume Oil According to the Invention

### Example P5

| | Ref-5 | P5 |
|---|---|---|
| CYCLOHEXADECENONE, (8E/Z)- + | 200 | 200 |
| CYCLOHEXADECENONE, (7E)- | | |
| MUGWORT OIL | 10 | 10 |
| BERGAMOT OIL | 60 | 60 |
| CEDRYLMETHYL ETHER | 40 | 40 |
| CISTUS OIL SPAN., 10% in DPG | 10 | 10 |
| LEMON OIL | 20 | 20 |
| COUMARIN | 40 | 40 |
| CYCLOHEXYLOXYALLYL ACETATE, 10% in DPG | 5 | 5 |
| MYRCENOL, DIHYDRO- | 50 | 50 |
| ETHANEDIOL BRASSYLATE | 120 | 120 |
| METHYL BENZOATE, 2,4-DIHYDROXY- | 20 | 20 |
| 3,6-DIMETHYL-, 10% in DPG | | |
| METHYL-DIHYDROJASMONATE | 30 | 30 |
| IRALDEIN, GAMMA- | 20 | 20 |
| BICYCLO[4.4.0]DECENE, 3,4,10,10-TETRAMETHYL-3- | 120 | 120 |
| HYDROXYETHYL-1 (6) | | |
| SPEARMINT OIL, 10% in DPG | 10 | 10 |
| LAVENDER OIL | 10 | 10 |
| LINALOOL | 20 | 20 |
| LINALYL ACETATE | 20 | 20 |
| INDENO[1.2-D]-M-DIOXIN, 2,4-DIMETHYL- | 90 | 90 |
| TETRAHYDRO- | | |
| MUSCATEL SAGE ABSOLUTE | 5 | 5 |
| CLOVE BLOSSOM OIL | 10 | 10 |
| VANILLIN | 20 | 20 |
| DIOXOL, 4H-4A,9-METHANOAZULENO(5,6-D)- | 20 | 0 |
| OCTAHYDRO-2,2,5,8,8,9A-HEXAMETHYL-, 1,3- | | |
| (Ambrocenide ®), 0.1% in DPG | | |
| Dioxatetracyclo[6.5.1.0^{1,10}.0^{3,7})tetradecane, 5,7,9,9,13- | 0 | 20 |
| pentamethyl-5-propyl-4,6-(compound of formula (II)), | | |
| 0.1% in DPG | | |
| TOTAL | 950 | 950 |

The comparative example (Ref-5) with Ambrocenide® has a very radiant and intensely lavender-like effect. In perfume oil P5, the compound of the formula (II) makes the whole composition more natural and lavender-like, and as a result it appears of higher quality with more volume.

### Perfume Oil According to the Invention

### Example P6

| | Ref-6 | P6 |
|---|---|---|
| HEXADECEN-1,16-OLIDE, 7 (9)Z- | 20 | 20 |
| CYCLOHEXADECENONE, (8E/Z)- + | 200 | 200 |
| CYCLOHEXADECENONE, (7E)- | | |
| INDAN-4-ONE, 1,1,2,3,3-PENTAMETHYL- | 20 | 20 |
| TETRAHYDRO- | | |
| ETHANEDIOL BRASSYLATE | 150 | 150 |
| 5-HEXENYL BUTYRATE, 2-METHYL- | 5 | 5 |
| PENTADECEN-1,15-OLIDE, (11E/Z)- + | 90 | 90 |
| PENTADECEN-1,15-OLIDE, 12E- | | |
| METHYL-DIHYDROJASMONATE | 210 | 210 |
| BICYCLO[4.4.0]DECENE, 3,4,10,10-TETRAMETHYL-3- | 200 | 200 |
| HYDROXYETHYL-1 (6) | | |
| CYCLOHEXANONE, 3,3,5,5-TETRAMETHYL-4- | 20 | 20 |
| (1-ETHOXYVINYL)- | | |
| PENTEN-2-OL, 3,3-DIMETHYL-5-(2,2,3-TRIMETHYL- | 5 | 5 |
| 3-CYCLOPENTENYL)-4- | | |
| ISOLONGIFOLANONE-ETHANEDIOL KETAL | 70 | 70 |
| DIOXOL, 4H-4A,9-METHANOAZULENO(5,6-D)- | 5 | 5 |
| OCTAHYDRO-2,2,5,8,8,9A-HEXAMETHYL-, 1,3- | | |
| (Ambrocenide ®), 0.1% in DPG | | |
| Dioxatetracyclo[6.5.1.0^{1,10}.0^{3,7})tetradecane, | 0 | 0 |
| 5,7,9,9,13-pentamethyl-5-propyl-4,6-(compound of formula | | |
| (II)), 0.1% in DPG | | |
| TOTAL | 995 | 995 |

The comparative example (Ref-6) with Ambrocenide® has a cool transparency coupled with a dry woodiness. In perfume oil P6 the compound of the formula (II) endows the whole composition with a pronounced harmony and warmth, wherein the fruitiness is intensified (pushed) to a fine plum note coupled with tobacco effects.

### Perfume Oil According to the Invention

### Example P7

| | Ref-7 | P7 |
|---|---|---|
| NAPHTHO[2.1-B]FURAN, DODECAHYDRO-3A.6.6.9A-TETRAMETHYL- | 5 | 5 |
| BENZYL SALICYLATE | 50 | 50 |
| BERGAMOT OIL | 50 | 50 |
| COUMARIN | 5 | 5 |
| DAMASCONE, TRANS-ALPHA-, 1% in DPG | 20 | 20 |
| DECALACTONE GAMMA, 10% in DPG | 15 | 15 |
| ETHANEDIOL BRASSYLATE | 30 | 30 |
| NONADIEN-3-OL, 3,7-DIMETHYL-1,6- | 60 | 60 |
| 3,7-DIME-1,6-NONADIEN-3-YL ACETATE | 50 | 50 |
| PYRANOL, 2-ISOBUTYL-4-METHYL-4-TETRAHYDRO-(E/Z) | 70 | 70 |
| 5-HEXENYL BUTYRATE, 2-METHYL- | 5 | 5 |
| BICYCLO[2.2.2]OCTENE, 6-ME-8-ISOPROPYL-2 (3)-(1,3-DIOXOLAN-2-YL | 5 | 5 |
| METHYL-DIHYDROJASMONATE | 280 | 280 |
| PROPANAL, 2-PIPERONYL- | 20 | 20 |
| BENZALDEHYDE, 3,4-METHYLENE DIOXY- | 10 | 10 |
| 2-(1-(3,3-DIMETHYLCYCLOHEXYL)-ETHOXY-2-METHYLPROPYL | 60 | 60 |
| PROPIONATE | | |
| 3Z-HEXENYL SALICYLATE | 5 | 5 |
| BICYCLO[4.4.0]DECENE, 3,4,10,10-TETRAME-3-HYDROXYETHYL-1 | 120 | 120 |
| (6) | | |
| PENTADECANOLIDE, 1,15- | 30 | 30 |
| MANDARIN OIL | 20 | 20 |
| CYCLOPENTADECENONE, 3-METHYL-5E- | 3 | 3 |
| BUTANOL, 2-METHYL-4-PHENYL-2- | 10 | 10 |
| PYRIDINE, 4-DECYL-, 1% in DPG | 2 | 2 |
| PENTEN-2-OL, 3,3-DIMETHYL-5-(2,2,3-TRIMETHYL-3- | 20 | 20 |
| CYCLOPENTENYL)-4- | | |
| CYCLOHEXANOL, 4-(3-METHYL-BUTYL)-(E/Z) | 15 | 15 |
| VANILLIN | 20 | 20 |
| DIOXOL, 4H-4A,9-METHANOAZULENO(5,6-D)-OCTAHYDRO- | 5 | 0 |
| 2,2,5,8,8,9A-HEXAMETHYL-, 1,3-(Ambrocenide ®), 0.1% in DPG | | |
| Dioxatetracyclo[6.5.1.0^{1,10}.0^{3,7})tetradecane, 5,7,9,9,13-pentamethyl-5- | 0 | 5 |
| propyl-4,6-(compound of formula (II)), 0.1% in DPG | | |
| DIPROPYLENE GLYCOL | 15 | 15 |
| TOTAL | 1000 | 1000 |

By replacing Ambrocenide® in the comparative example (Ref-7) with the compound of the formula (II), the whole composition P7 has greater volume and is more elegant and therefore also of higher quality, perfume oil P7 has a more complex effect and the coumarin note is harmonized very well.

### Perfume Oil According to the Invention

### Example P8

| | Ref-8 | P8 |
|---|---|---|
| Ethylacetoacetate | 60 | 60 |
| Propanal, 2-methyl-2-(4-ethylbenzyl)- | 25 | 25 |
| Cyclohexene, 2,4-dimethyl-1-formyl-3-(E/Z) | 15 | 15 |
| Hexenol cis-3 | 130 | 130 |
| 3Z-Hexenylmethyl carbonate | 20 | 20 |
| Cyclohexyloxy allyl acetate | 25 | 25 |
| Benzene methanol, .alpha.-methyl-, 1-acetate | 40 | 40 |
| Linalyl acetate | 80 | 80 |
| Myrcenol, dihydro- | 650 | 650 |
| Mandarin oil | 260 | 260 |
| Grapefruit oil | 260 | 260 |
| Hexenaldimethylacetal, 2,2,5-trimethyl-4- | 170 | 170 |
| Methylanthranilate, 10% DPG | 155 | 155 |
| Cardamom oil | 5 | 5 |
| Red Berry Extract | 25 | 25 |
| Nutmeg oil | 20 | 20 |
| Ethyl butyrate, 10% DPG | 10 | 10 |
| Undecalactone, gamma- | 65 | 65 |
| Ethyl pentanoate, 2-methyl-, 10% DPG | 15 | 15 |
| 2-Propen-1-yl heptanoate | 40 | 40 |
| Ethyl maltol | 15 | 15 |
| Benzene propanal, alpha-methyl-4-(1,1-dimethylethyl)- | 250 | 250 |
| 1,3-Benzodioxol-5-propanal, alpha-methyl- | 200 | 200 |
| Hydroxycitronellal | 200 | 200 |
| 4-Methyl-2-(2-methylpropyl)oxan-4-ol | 260 | 260 |
| 1,6-Octadien-3-ol, 3,7-dimethyl- | 40 | 40 |
| Nonadien-3-ol, 3,7-dimethyl-1,6- | 130 | 130 |
| 1,1-Dimethyl-2-phenylethyl butyrate | 65 | 65 |
| Phenylethyl alcohol | 40 | 40 |
| 2-Phenoxyethyl propionate, 2-methyl- | 40 | 40 |
| 2-Buten-1-one, 1-(2,6,6-trimethyl-2-cyclohexen-1-yl)-(E) | 40 | 40 |
| 2-Buten-1-one, 1-(2,6,6-trimethyl-1,3-cyclohexadien-1-yl)- | 5 | 5 |
| Methyl-dihydrojasmonate | 390 | 390 |
| 3-Buten-2-one, 3-methyl-4-(2,6,6-trimethyl-2-cyclohexen-1-yl)- | 220 | 220 |
| Vanillin | 230 | 230 |
| Cinnamon Bark Oil | 15 | 15 |
| 2H-1-Benzopyran-2-one | 200 | 200 |
| Cyclohexanol acetate, 4-(1,1-dimethylethyl)-(Z) | 50 | 50 |
| Cedarwood Oil | 15 | 15 |
| Bicyclo[4.4.0]decene, 3,4,10,10-tetramethyl-3-hydroxyethyl-1 (6) | 1.500 | 1.500 |
| Cyclododecane, (ethoxymethoxy)- | 300 | 300 |
| 2-Buten-1-ol, 2-ethyl-4-(2,2,3-trimethyl-3-cyclopenten-1-yl)-(2E) | 130 | 130 |
| Dioxol, 4H-4a,9-methanoazuleno(5,6-d)-octahydro-2,2,5,8,8,9a- | 30 | |
| hexamethyl-, 1,3-(Ambrocenide ®), 10.0% in DPG | | |
| Dioxatetracyclo[6.5.1.0^{1,10}.0^{3,7})tetradecane, 5,7,9,9,13-pentamethyl-5- | | 30 |
| propyl-4,6-(compound of formula (II)), 10.0% in DPG | | |
| 5H-3.5a-Epoxynaphth[2.1-c]oxepine, dodecahydro-3,8,8,11a-tetramethyl- | 1.200 | 1.200 |
| Naphtho[2,1-b]furan, dodecahydro-3a,6,6,9a-tetramethyl- | 220 | 220 |
| Dipropylene glycol | 175 | 175 |
| TOTAL | 8.030 | 8.030 |

The comparative example Ref-8 with Ambrocenide® is characterized by the contradiction between fresh woody notes and sweet balsamic aspects. Replacing Ambrocenide® with the compound of formula (II) gives the whole composition more impact, volume, character coupled with velvety-powdery aspects.

### Perfume Oil According to the Invention

### Example P9

| | Ref-9 | P9 |
|---|---|---|
| Undecenal, 2,6,10-trimethyl-9- | 15.00 | 15.00 |
| Propanal, 2-methyl-2-(4-ethylbenzyl)- | 3.00 | 3.00 |
| Cis-3 hexenyl acetate | 3.00 | 3.00 |
| Cyclohexene, 2,4-dimethyl-1-formyl-3-(E/Z) | 2.00 | 2.00 |
| Tricyclo(5.2.1.0)decane, 8-formyl- | 2.00 | 2.00 |
| Cyclohexyloxyallyl acetate | 4.00 | 4.00 |
| Heptenal, 2,6-dimethyl-5- | 2.00 | 2.00 |
| Identoil ® bergamot | 45.00 | 45.00 |
| Myrcenol, dihydro- | 90.00 | 90.00 |
| 2,6-Octadienal, 3,7-dimethyl-(2E/Z) | 10.00 | 10.00 |
| 2-Pentene nitrile, 3-methyl-5-phenyl-(2E/Z) | 3.00 | 3.00 |
| Orange oil | 30.00 | 30.00 |
| Hexenaldimethylacetal, 2,2,5-trimethyl-4- | 2.00 | 2.00 |
| Lavandin oil | 10.00 | 10.00 |
| Cardamom oil | 1.00 | 1.00 |
| Ethyl pentanoate, 2-methyl-, 10% DPG | 10.00 | 10.00 |
| Benzene propanal, alpha-methyl-4-(1,1-dimethylethyl)- | 40.00 | 40.00 |
| 2H-1,5-benzodioxepin-3(4H)-one, 7-methyl- | 20.00 | 20.00 |
| Benzene propanol, beta,beta.3-trimethyl- | 40.00 | 40.00 |
| Geranium oil | 2.00 | 2.00 |
| 3-Buten-2-one, 1-(2,4,4-trimethyl-2-cyclohexen-1- | 10.00 | 10.00 |
| yl)-(2E) | | |
| alpha-Hexylcinnamaldehyde | 60.00 | 60.00 |
| Cyclopentane methyl acetate, 3-oxo-2-pentyl-(E) | 90.00 | 90.00 |
| Propane nitrile, 3-(3-hexenyloxy)-(Z) | 2.00 | 2.00 |
| 3-Buten-2-one, 4-(2,6,6-trimethyl-1-cyclohexen-1-yl)- | 5.00 | 5.00 |
| Benzene, 1-methoxy-4-(1-propenyl)-(E) | 10.00 | 10.00 |
| Ethanone, 1-(2-benzofuranyl)- | 2.00 | 2.00 |
| Cyclohexanol acetate, 4-(1,1-dimethylethyl)-(Z) | 30.00 | 30.00 |
| Cedarwood Oil | 15.00 | 15.00 |
| Cyclohexane propanol, alpha-ethyl-2,2,6-trimethyl- | 30.00 | 30.00 |
| (E/Z) | | |
| Bicyclo[4.4.0]decen, 3,4,10,10-tetramethyl-3- | 100.00 | 100.00 |
| hydroxyethyl-1 (6) | | |
| Cyclododecane, (ethoxymethoxy)- | 30.00 | 30.00 |
| Patchouli oil | 10.00 | 10.00 |
| 2-Buten-1-ol, 2-ethyl-4-(2,2,3-trimethyl-3- | 30.00 | 30.00 |
| cyclopenten-1-yl)-(2E) | | |
| Methyl benzoate, 2,4-dihydroxy-3,6-dimethyl- | 3.00 | 3.00 |
| Tetramethyl dodecahydro-3a,6,6,9a-naphtho(2,1- | 12.00 | 12.00 |
| b)furan | | |
| Isolongifolanone ethanediol ketal | 50.00 | 50.00 |
| Dioxol, 4H-4a,9-methanoazuleno(5,6-d)-octahydro- | 2.00 | |
| 2,2,5,8,8,9a-hexamethyl-, 1,3-(Ambrocenide ®), | | |
| 10.0% in DPG | | |
| Dioxatetracyclo[6.5.1.0^{1,10}.0^{3,7})tetradecane, | | 2.00 |
| 5,7,9,9,13-pentamethyl-5-propyl-4,6-(compound of | | |
| formula (II)), 10.0% in DPG | | |
| Pentadecanolide, 1,15- | 30.00 | 30.00 |
| Cyclohexadecenone, 8E/Z- | 120.00 | 120.00 |
| Dipropylene glycol | 25.00 | 25.00 |
| TOTAL | 1000.00 | 1000.00 |

The comparative example Ref-9 with Ambrocenide® is a modern Fougere type with an aqueous citrus top note and a woody base. By replacing Ambrocenide® with the compound of the formula (II), the white wood, and cedarwood and sandalwood aspect is intensified.

### Perfume Oil According to the Invention

### Examples P10

| | Ref-10 | P10 |
|---|---|---|
| Undecenal, 2,6,10-trimethyl-9- | 8 | 8 |
| 10-Undecenal | 8 | 8 |
| Cyclohexene, 2,4-dimethyl-1-formyl-3-(E/Z) | 5 | 5 |
| 3Z-Hexenylmethyl carbonate | 8 | 8 |
| Indeno[1,2-d]-1,3-dioxin, 4.4a.5.9b-tetrahydro-2,4- | 70 | 70 |
| dimethyl- | | |
| Cyclohexanol acetate, 3,3,5-trimethyl- | 20 | 20 |
| Orange oil | 20 | 20 |
| Methyl Naphthyl Ketone Beta | 5 | 5 |
| Ethanone, 1-(3-methyl-2-benzofuranyl)- | 2 | 2 |
| 2-Butanone, 4-(4-hydroxyphenyl)- | 1 | 1 |
| Peach Base | 8 | 8 |
| Pyranol, 2-isobutyl-4-methyl-4-tetrahydro-(E/Z) | 10 | 10 |
| 1.6-Octadien-3-ol, 3,7-dimethyl- | 90 | 90 |
| 2H-Pyran, tetrahydro-2-methyl-2-(2-methyl-1- | 3 | 3 |
| propenyl)-(E/Z) | | |
| 2-Phenylethanol | 70 | 70 |
| Benzene pentanol, beta-methyl- | 25 | 25 |
| 2,6-Octadien-1-olacetate, 3,7-dimethyl-(2E/Z) | 30 | 30 |
| But-2-en-1-one, 1-(2,6,6-trimethyl-cyclohex-3-enyl)-(E) | 3 | 3 |
| Propyl benzoate | 10 | 10 |
| Methyl-dihydrojasmonate (E) | 70 | 70 |
| Amyl salicylate N/iso | 17 | 17 |
| Benzyl salicylate | 80 | 80 |
| Cis-3 hexenyl salicylate | 10 | 10 |
| Hexyl benzoate, 2-hydroxy- | 33 | 33 |
| 3-Decen-5-ol, 4-methyl- | 5 | 5 |
| Orris Base | 5 | 5 |
| 3-Buten-2-one, 3-methyl-4-(2,6,6-trimethyl-2- | 80 | 80 |
| cyclohexen-1-yl)- | | |
| Benzaldehyde, 3,4-methylene dioxy- | 10 | 10 |
| Vanillin | 4 | 4 |
| Cinnamon alcohol | 4 | 4 |
| 2H-1-Benzopyran-2-one | 8 | 8 |
| Acetylcedrene | 70 | 70 |
| Cyclododecane, (ethoxymethoxy)- | 40 | 40 |
| 2-Pentanone, 4-cyclohexyl-4-methyl- | 8 | 8 |
| Patchouli Base | 37 | 37 |
| Patchouli oil | 33 | 33 |
| Dioxol, 4H-4a,9-methanoazuleno(5,6-d)-octahydro- | 20 | |
| 2,2,5,8,8,9a-hexamethyl-, 1,3-(Ambrocenide ®), | | |
| 1.0% in DPG | | |
| Dioxatetracyclo[6.5.1.0^{1,10}.0^{3,7})tetradecane, | | 2 |
| 5,7,9,9,13-pentamethyl-5-propyl-4,6-(compound | | |
| of formula (II) 10.0% in DPG | | |
| Pentadecen-1,15-olide, (11E/Z)- + pentadecen-1,15- | 40 | 40 |
| olide, 12E- | | |
| Triethyl citrate | 30 | 48 |
| TOTAL | 1000 | 1000 |

The comparative example Ref-10 with Ambrocenide® is a floral Chypre type with an aldehyde-like top note, coupled with floral heart notes and rounded-off by patchouli and musk. Replacing Ambrocenide® with the compound of the formula (II) gives the whole composition a more beneficial, warmer and more voluminous character, and in addition adherence is improved.

The perfume oils P1, P2, P3, P4, P5, P6, P7, P8, P9 and P10 from the above perfume oil examples were in each case incorporated separately in the following formulations.

The olfactory effects described above for the respective perfume oil were in each case also observed in the following formulations.

### EXAMPLES OF FORMULATIONS

### Example F1

### Washing Powder

| Material | Chemical name | Function | wt. % | wt. % |
|---|---|---|---|---|
| Sodium | Sodium metasilicate | | to | to |
| metasilicate | pentahydrate | | 100 | 100 |
| pentahydrate | | | | |
| Sodium hydrogen | Sodium hydrogen | Alkali | 15.0 | 15.0 |
| carbonate | carbonate | | | |
| Sodium | Sodium carbonate | Bleach | 15.0 | 15.0 |
| percarbonate | peroxyhydrate | | | |
| Peractive AC | TAED/Na- | Activator | 5.00 | 5.00 |
| Blue | carboxymethylcellulose | | | |
| Genapol OA-080 | Oxoalcohol C14-15, | Non-ionic | 3.00 | 3.00 |
| | 8EO | surfactant | | |
| Texapon K12 | Sodium lauryl | Anionic | 7.00 | 7.00 |
| powder | sulfate C12 | surfactant | | |
| Tinopal CBS-X | | Brightener | 0.50 | 0.50 |
| Savinase 6.0 T, | Protease | Enzyme | 0.40 | 0.40 |
| Type W | | | | |
| Termamyl 120 T | Alpha-amylase | Enzyme | 0.30 | 0.30 |
| Sodium sulfate | Sodium sulfate | Filler | 5.50 | 5.50 |
| Perfume oil P1, | | Perfume | 0.30 | 0.50 |
| P2, P3, P4, P5, | | (fragrance) | | |
| P6, P7, P8, P9 or | | | | |
| P10 | | | | |

### Example F2

### All-Purpose Cleaner

| | Chemical | | | |
|---|---|---|---|---|
| Material | name | Function | wt. % | wt. % |
| Deionized | Water | Solvent | to 100 | to 100 |
| water | | | | |
| Mergal K9N | 5-Chloro-2- | Preservative | 0.1 | 0.1 |
| | methyl-3- | | | |
| | (2H)- | | | |
| | isothiazolone | | | |
| | and 2- | | | |
| | methyl-3- | | | |
| | (2H)- | | | |
| | isothiazolone | | | |
| Trisodium | Trisodium | Complexing agent | 3.0 | 3.0 |
| citrate | citrate | | | |
| dihydrate | dihydrate | | | |
| Zetesol NL-2 | Fatty alcohol | Anionic surfactant | 30.0 | 30.0 |
| | C12-14- | | | |
| | sulfate, | | | |
| | sodium | | | |
| Imbentin | Fatty alcohol | Non-ionic surfactant | 5.0 | 5.0 |
| C/125/055 | C12-C15, | | | |
| | 8EO | | | |
| Ethanol | Ethanol | Solvent | 2.0 | 2.0 |
| Perfume oil | | Perfume (fragrance) | 0.3 | 0.5 |
| P1, P2, P3, | | | | |
| P4, P5, P6, | | | | |
| P7, P8, P9 or | | | | |
| P10 | | | | |

### Example F3

### Shampoo

| Material | INCI name | wt. % | wt. % |
|---|---|---|---|
| Deionized | Water | to 100 | to 100 |
| water | | | |
| Plantacare | Sodium laureth sulfate, | 20.0 | 20.0 |
| PS 10 | lauryl glucoside | | |
| Euperlan PK | Glycol distearate, sodium | 6.0 | 6.0 |
| 771 | lauryl sulfate, cocamide | | |
| | MEA, laureth-10 | | |
| Dragocid | Phenoxyethanol, | 0.5 | 0.5 |
| Liquid | methylparaben, | | |
| | ethylparaben, butylparaben, | | |
| | propylparaben, | | |
| | isobutylparaben | | |
| Sodium | Sodium chloride | 1.4 | 1.4 |
| chloride | | | |
| Citric acid | Citric acid | 0.1 | 0.1 |
| monohydrate | | | |
| crystalline | | | |
| Perfume oil | Perfume (fragrance) | 0.5 | 0.8 |
| P1, P2, P3, | | | |
| P4, P5, P6, | | | |
| P7, P8, P9 or | | | |
| P10 | | | |

### Example F4

### Shower Gel

| Material | INCI name | wt. % | wt. % |
|---|---|---|---|
| Deionized | Water | to 100 | to 100 |
| water | | | |
| Plantacare PS | Sodium laureth sulfate, | 20.0 | 20.0 |
| 10 | lauryl glucoside | | |
| Dragocid | Phenoxyethanol, | 0.5 | 0.5 |
| Liquid | methylparaben, | | |
| | ethylparaben, butylparaben, | | |
| | propylparaben, | | |
| | isobutylparaben | | |
| Sodium | Sodium chloride | 1.4 | 1.4 |
| chloride | | | |
| Citric acid | Citric acid | 1.3 | 1.3 |
| monohydrate | | | |
| crystalline | | | |
| Perfume oil | Perfume (fragrance) | 0.5 | 0.7 |
| P1, P2, P3, | | | |
| P4, P5, P6, | | | |
| P7, P8, P9 or | | | |
| P10 | | | |

### Example F5

### Fabric Softener

| Material | Chemical name | Function | wt. % | wt. % |
|---|---|---|---|---|
| Deionized | Water | Solvent | to | to |
| water | | | 100 | 100 |
| Rewoquat | Dialkylesterammonium- | Cationic | 16.6 | 16.6 |
| WE 18 | ethosulfate | surfactant | | |
| Mergal K9N | 5-Chloro-2-methyl-3- | Preservative | 0.10 | 0.10 |
| | (2H)-isothiazolone and | | | |
| | 2-methyl-3-(2H)- | | | |
| | isothiazolone | | | |
| Dow Corning | Polydimethyl-siloxane | Antifoaming | 0.30 | 0.30 |
| 1520 | | agent | | |
| Antifoam | | | | |
| Magnesium | Magnesium chloride | Consistency | 10.00 | 10.00 |
| chloride 1% | solution | agent | | |
| solution | | | | |
| Perfume oil | | Fragrance | 0.55 | 0.75 |
| P1, P2, P3, | | | | |
| P4, P5, P6, | | | | |
| P7, P8, P9 or | | | | |
| P10 | | | | |

### Example F6

### Eau De Cologne/Eau De Toilette

| Ingredients | wt. % | wt. % |
|---|---|---|
| Perfume oil P1, P2, P3, P4, P5, P6, P7, P8, P9 or | 5 | 10 |
| P10 | | |
| Ethanol | to 100 | to 100 |
| Water | 18 | 10 |

### Example F7

### Aerosol Pump Spray

| Ingredients | wt. % | wt. % |
|---|---|---|
| Perfume oil P1, P2, P3, P4, P5, P6, P7, P8, | 1.0 | 1.5 |
| P9 or P10 | | |
| Ethanol | to 100 | to 100 |
| Water | 5.0 | 8.0 |
| Alpha-tocopherol | 0.20 | 0.20 |
| Hydroxypropylcellulose | 0.20 | — |
| Rosemary extract, ethanol-soluble | 0.22 | — |
| Cetyl alcohol | 1.00 | 0.50 |

### Example F8

### Shampoo

| Ingredients | wt. % | wt. % | wt. % |
|---|---|---|---|
| Sodium lauryl ether sulfate (e.g. | 12 | 12 | 12 |
| Texapon NSO, from Cognis Germany | | | |
| GmbH) | | | |
| Cocamidopropylbetaine (e.g. | 2 | 2 | 2 |
| Dehyton K, from Cognis Germany | | | |
| GmbH) | | | |
| Sodium chloride | 1.4 | 1.4 | 1.4 |
| Citric acid | 1.3 | 1.3 | 1.3 |
| Phenoxyethanol, methyl-, ethyl-, | 0.5 | 0.5 | 0.5 |
| butyl- and propylparaben | | | |
| Perfume oil P1, P2, P3, P4, P5, P6, | 0.3 | 0.5 | 0.7 |
| P7, P8, P9 or P10 | | | |
| Water | to 100 | to 100 | to 100 |

### Example F9

### Washing Powder

| Ingredients | wt. % | wt. % | wt. % |
|---|---|---|---|
| Linear Na-alkylbenzene sulphonate | 8.8 | 8.8 | 8.8 |
| Ethoxylated fatty alcohol C12-18 (7 EO) | 4.7 | 4.7 | 4.7 |
| Na-soap | 3.2 | 3.2 | 3.2 |
| Antifoaming agent DOW CORNING(R) | 3.9 | 3.9 | 3.9 |
| 2-4248S POWDERED ANTIFOAM, | | | |
| silicone oil on zeolite as carrier material | | | |
| Zeolite 4A | to 100 | to 100 | to 100 |
| Na-carbonate | 11.6 | 11.6 | 11.6 |
| Na-salt of a copolymer of acrylic and | 2.4 | 2.4 | 2.4 |
| maleic acid (Sokalan CP5) | | | |
| Na-silicate | 3.0 | 3.0 | 3.0 |
| Carboxymethylcellulose | 1.2 | 1.2 | 1.2 |
| Dequest 2066 | 2.8 | 2.8 | 2.8 |
| ([[(phosphonomethyl)imino]bis[(ethylene | | | |
| nitrilo)bis(methylene)]]tetrakis-phosphonic | | | |
| acid, sodium salt) | | | |
| Optical brightener | 0.2 | 0.2 | 0.2 |
| Na-sulfate | 6.5 | 6.5 | 6.5 |
| Protease | 0.4 | 0.4 | 0.4 |
| Sodium perborate tetrahydrate | 21.7 | 21.7 | 21.7 |
| Perfume oil P1, P2, P3, P4, P5, P6, P7, | 0.25 | 0.35 | 0.5 |
| P8, P9 or P10 | | | |
| EDTA | 1.0 | 1.0 | 1.0 |

### Example F10

### Liquid Detergent

| Ingredients | wt. % |
|---|---|
| Deionized water | 39.9 |
| Optical brightener | 0.10 |
| Coconut fatty acids (C12-C18) | 7.5 |
| Potassium hydroxide 50% solution | 4.3 |
| Propane-1,2-diol | 5.00 |
| Fatty alcohols C12-C15, 8 EO | 12.0 |
| Na-salt of secondary alkylsulphonates (C13-C17) | 17.0 |
| Triethanolamine | 2.0 |
| Trisodium citrate dihydrate | 5.0 |
| Dequest 2066 | 3.0 |
| ([[(phosphonomethyl)imino]bis[(ethylene nitrilo)bis | |
| (methylene)]]tetrakis-phosphonic acid, sodium salt) | |
| Ethanol | 3.0 |
| Enzymes | 0.7 |
| Perfume oil P1, P2, P3, P4, P5, P6, P7, P8, P9 or | 0.5 |
| P10 | |

### Example F11

### Liquid Detergent Concentrate

| Ingredients | wt. % |
|---|---|
| Deionized water | 13.4 |
| Coconut fatty acids (C12-C18) | 10.0 |
| Fatty alcohols C12-C15, 8 EO | 26.0 |
| Na-salt of secondary alkylsulphonates (C13-C17) | 26.5 |
| Triethanolamine | 8.5 |
| Na-salt of fatty alcohol sulfates C12-C14 | 3.0 |
| Ethanol | 5.5 |
| Urea | 4.5 |
| Enzymes | 0.9 |
| Citric acid | 1.0 |
| Perfume oil P1, P2, P3, P4, P5, P6, P7, P8, P9 or | 0.7 |
| P10 | |

### EXAMPLES OF APPLICATION

### Example of Intrinsic Adhesion

### Compound of the Formula (II) Versus Ambrocenide®

The perfume to be investigated in each case is dipped as 10% solution in DPG on coded odor strips, i.e. the perfume to be investigated is applied to the odor strip by dipping the respective odor strip in the respective solution of the perfume to be investigated, and immediately thereafter is submitted to olfactory assessment at the following time intervals:

1 hour; 3 hours; 10 hours; 1 day; 3 days; 10 days; 1 month; 3 months. The testers assess the odor intensity on a scale from 1=odorless to 9=very strong.

Testing is continued until the testers' mean value of intensity falls below a value of 1.5 (very faint). If 50% of the testers assign an intensity of 1, and thus indicate that the perfume applied on the odor strip is no longer perceived, the test is declared completed. Three months is the maximum duration of testing.

| | Compound of the | |
|---|---|---|
| | formula (II), 10% in DPG | Ambrocenide ®, 10% in DPG |
| Intrinsic | more than 3 months | 10 days |
| adhesion | | |

The compound of formula (II) shows far longer adhesion on the odor strip than Ambrocenide®.

### Example for Substantivity on Hair

### Compound of the Formula (II) Versus Ambrocenide®

The perfume to be investigated is incorporated in an amount of 0.6 wt. % as 50% solution in DPG in a shampoo formulation as in example F8 instead of the perfume oil used there. Two strands of hair are required for each perfume to be investigated. In addition, as reference, in each case one pair of strands of hair is washed with the unperfumed shampoo formulation of example F8.

All the strands of hair are washed together in a 2-litre beaker with neutral shampoo (soak for min. 2 h). Then the strands are rinsed well under running water and then dried at room temperature (approx. 23° C.). 100 ml of 20% aqueous solution is prepared from each shampoo (also unperfumed mass). Now for each shampoo, the strands of one pair are washed together for 2 min in the solution. Next, these two strands are rinsed together for 20 seconds under hand-hot running water. Both strands of hair are combed. One strand of hair is wrapped wet in aluminum foil. The second strand of hair is dried with a hairdryer.

A testing panel assesses the prepared strands of hair by sniffing. The testers assess the intensity of the samples on a scale from 0 (odorless) to 6 (very strong).

### Sequence:

a) freshly washed, dry hair
b) freshly washed hair, wet

| Substantivity on | | |
|---|---|---|
| hair | Compound of formula (II) | Ambrocenide ® |
| Wet hair | 1.4 | 1.2 |
| Dry hair | 1.7 | 0.5 |

The intensity value of the compound of the formula (II), on dry hair, is more than 3 times higher compared with Ambrocenide®.

### Example of Substantivity on Cotton

### Compound of Formula (II) Versus Ambrocenide®

The perfume to be investigated is incorporated in an amount of 0.5 wt. % as 50% solution in DPG in a fabric softener formulation as in example F5 instead of the perfume oil used there.

Cotton cloths are washed at 95° C. in a washing machine until neutral and are dried in the open at room temperature.

In each case two identically coded cotton cloths are rinsed with 370 g of 1% aqueous fabric softener solution containing the perfume to be investigated in each case in a pot of the Linitest machine in the soft-rinse program. Then the solution in the individual pots is poured away, the cloths from each pot are wrung out and all the pairs of cloths are spun-dry as a pair for 20 seconds. Then from each pair of cloths, one cloth is wrapped up wet, and one is hung up to dry.

A testing panel assesses the prepared samples by sniffing.

The testers assess the intensity of the samples on a scale from 0 (odorless) to 6 (very strong).

It starts in each case with the unperfumed reference and then the samples.

### Sequence:

a) dry cloth
b) wet cloth

| Substantivity on cotton | Compound of formula (II) | Ambrocenide ® |
|---|---|---|
| Wet laundry | 1.6 | 1.2 |
| Dry laundry | 1.5 | 0.8 |

The intensity value of the compound of the formula (II), on the dry cloth, is higher by a factor of 2 compared with Ambrocenide®, and the intensity of the wet cloth is also assessed as higher for the compound of the formula (II).

Furthermore, the dry cotton cloths were stored for a longer period of time and in each case were assessed after a plurality of days as described above.

| | | |
|---|---|---|
| Day 3 | Compound of formula (II) | 2.00 |
| | Ambrocenide ® | 1.20 |
| Day 8 | Compound of formula (II) | 1.79 |
| | Ambrocenide ® | 0.93 |
| Day 10 | Compound of formula (II) | 1.80 |
| | Ambrocenide ® | 1.10 |
| Day 14 | Compound of formula (II) | 1.69 |
| | Ambrocenide ® | 0.92 |
| Day 18 | Compound of formula (II) | 1.80 |
| | Ambrocenide ® | 0.50 |

Even after a period of 18 days, the intensity of the compound of the formula (II) is still assessed as much stronger compared with Ambrocenide®.

### Example of Yield

### Compound of the Formula (I) Versus Compound of the Formula (II)

The test perfume was in each case diluted as 10% solution in DPG (corresponding to stage 1) with diethyl phthalate (DEP) in dilution steps, so that further-diluted solutions of the perfume to be tested in each case, of dilution stages 2 to 7, were obtained.

| Stage 1 | Stage 2 | Stage 3 | Stage 4 | Stage 5 | Stage 6 | Stage 7 |
|---|---|---|---|---|---|---|
| 10% in | 1% in | 0.1% in | 0.01% | 0.001% | 0.0001% | 0.00001% |
| DPG | DPG | DPG | in DPG | in DPG | in DPG | in DPG |
| | and | and | and | and | and DEP | and DEP |
| | DEP | DEP | DEP | DEP | | |

The individual solutions are applied on odor strips by dipping and are assessed by a testing panel by sniffing. The testers sniff and assess starting from the lowest concentration reached, i.e. beginning with the dilution of stage 7.

The testers note the dilution stage in which a sensory difference is perceptible.

| | Compound of formula (I) | Compound of formula (II) |
|---|---|---|
| Yield | 2 | 4 |

In comparison with the compound of the formula (I), the compound of the formula (II) is still perceptible at a concentration that is 100 times lower.

### Example of Intensity of the Compound of the Formula (I) Versus the Compound of the Formula (II)

The sample to be investigated in each case is applied as 10% solution in DPG on an odor strip by dipping. A testing panel assesses the sample by sniffing. The testers assess the intensity of the compounds on a scale from 1 (odorless) to 9 (very strong).

| | Compound of formula (I), | Compound of formula (II), 10% |
|---|---|---|
| | 10% in DPG | in DPG |
| Intensity | 2 | 6 |

The compound of the formula (I) is only very faintly perceptible, whereas the compound of the formula (II) is perceived much more strongly.

### Example of Intensity in Air

### Compound of the Formula (I) Versus Compound of the Formula (II)

The compounds to be assessed are prepared in odor-neutral, heat-resistant plastic bags and then assessed.

The testers assess the intensity in air of the compounds to be assessed on a scale from 1 (odorless) to 9 (very strong).

| | Compound of formula (I) | Compound of formula (II) |
|---|---|---|
| Intensity in air | 1 | 6 |

The compound of formula (I) is described as odorless, whereas the compound of the formula (II) is assessed as very much stronger.

1. A compound of formula (II)

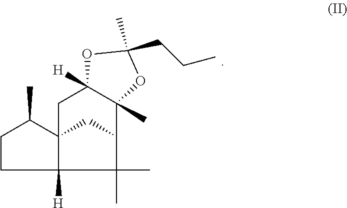

2. A mixture, comprising
(i) a compound of formula (I) 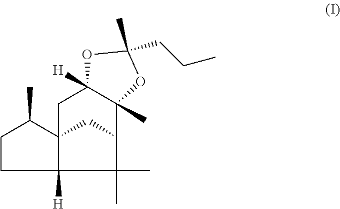
and
(ii) the compound of formula (II) as defined in claim 1,
wherein,
the weight ratio of the compound of formula (II) to the compound of formula (I) is in the range from 4:1 to 1:10,
and/or
the total proportion of the compounds of formulae (I) and (II) together is greater than 40 wt. %, relative to a total amount of 3,6,8,8-tetramethyl-octahydro-3a,7-methano-azulene-5,6-diol-methyl-n-propyl ketals contained in the mixture.

3. A fragrance substance, an agent for increasing the substantivity and/or retention of a fragrance mixture, or a fixative comprising the compound as claimed in claim 1.

4. A fragrance mixture, comprising
the compound of formula (II) as defined in claim 1
and
at least one further fragrance substance, wherein the further fragrance substance is not a 3,6,8,8-tetramethyl-octahydro-3a,7-methano-azulene-5,6-diol-methyl-n-propyl ketal,
wherein
(i) the amount of the compound of formula (II) is sufficient to impart an ambergris note and/or woody note,
and/or
(ii) the further fragrance substance imparts at least one note selected from the group consisting of fruity, flowery, spicy, woody, musk and ambergris-like and the amount of the compound of formula (II) is sufficient to modify and/or intensify at least one fruity, flowery, spicy, woody, musk or ambergris-like note,
and/or
(iii) the amount of the compound of formula (II) is sufficient to provide the fragrance mixture with an impression of volume, complexity, elegance and/or naturalness,
and/or
(iv) the amount of compound of formula (II) is sufficient to create, in comparison with an otherwise identically constituted comparative fragrance composition without the compound of formula (II), a more grooming, more harmonic, higher-quality and/or more natural odor impression.

5. The fragrance mixture as claimed in claim 4, wherein the amount of compound of formula (II) is in the range from 0.0001 to 25 wt. %, in each case relative to the total weight of the fragrance mixture.

6. The fragrance mixture as claimed in claim 4, comprising
(a) at least one further woody and/or ambergris fragrance substance,
and/or
(b) at least one musk fragrance substance.

7. The fragrance mixture as claimed in claim 6, wherein the musk fragrance substance of constituent (b) is selected from the group consisting of macrocyclic musk fragrance substances, nitro-musk fragrance substances, polycyclic musk fragrance substances and alicyclic musk fragrance substances.

8. The fragrance mixture as claimed in claim 6, wherein the musk fragrance substance of constituent (b) is selected from polycyclic and/or macrocyclic musk fragrance substances.

9. The fragrance mixture as claimed in claim 5, wherein the mass ratio of the total amount of musk fragrance substances of constituent (b) to compound (II) is greater than or equal to 10:1.

10. A method of intensifying an odor comprising a fruity, flowery, spicy, woody, musk and/or ambergris-like note, comprising:
mixing at least one fragrance substance with at least one fruity, flowery, spicy, woody, musk or ambergris-like note,
(i) wherein the amount of compound (II) as defined in claim 1 is sufficient to stress the odor impression of the fragrance substance or fragrance substances that produce at least one fruity, flowery, spicy, woody, musk or ambergris-like note,
or
(ii) wherein the amount of fragrance mixture as claimed in claim 4, is sufficient to stress the odor impression of the fragrance substance that produces at least one fruity, flowery, spicy, woody, musk or ambergris-like note.

11. A perfumed product comprising compound (II) as claimed in claim 1.

12. The perfumed product as claimed in claim 11, wherein the product further comprises at least one surfactant.

13. The perfumed product as claimed in claim 11, selected from the group consisting of washing or cleaning agent and hygiene or care products.

14. A method of providing (a) hair or (b) textile fibers with at least one fruity, flowery, spicy, woody, musk or ambergris-like odor note comprising:
preparing a fragrance mixture as claimed in claim 4; and
applying the mixture to (a) the hair or (b) the textile fibers.

15. The mixture according to claim 2, wherein
the weight ratio of the compound of formula (II) to the compound of formula (I) is in the range from 1:1 to 1:3,
and/or
the total proportion of the compounds of formulae (I) and (II) together is greater than 95 wt. %, relative to a total amount of 3,6,8,8-tetramethyl-octahydro-3a,7-methano-azulene-5,6-diol-methyl-n-propyl ketals contained in the mixture.

16. The fragrance mixture as claimed in claim 5, wherein the amount of compound of formula (II) is in the range from 0.001 to 15 wt. %, relative to the total weight of the fragrance mixture.

17. The fragrance mixture as claimed in claim 5, wherein the mass ratio of the total amount of musk fragrance substances of constituent (b) to compound (II) is greater than or equal to 50:1 to 100 000:1.

18. The fragrance mixture as claimed in claim 8, wherein the musk fragrance substance of constituent (b) is selected from the group consisting of macrocyclic C₁₄-C₁₈ ketones and macrocyclic C₁₄-C₁₈ lactones.

19. The perfumed product according to claim 11, comprising a sensorially effective amount of the compound of formula (II).
